(19) United States
(12) Patent Application Publication
(10) Pub. No.: US 2019/0046102 A1
(43) Pub. Date: Feb. 14, 2019

(54) PORTABLE FLUID MONITORING FOB AND METHODS FOR ACCURATELY MEASURING FLUID OUTPUT

(71) Applicant: The Trustees of Columbia University in the City of New York, New York, NY (US)

(72) Inventors: Alexander Kushnir, Passaic, NJ (US); Sinisa Vukelic, New York, NY (US)

(21) Appl. No.: 16/120,538

(22) Filed: Sep. 4, 2018

Related U.S. Application Data
(63) Continuation of application No. PCT/US2017/020981, filed on Mar. 6, 2017.
(60) Provisional application No. 62/303,961, filed on Mar. 4, 2016.
(60) Provisional application No. 62/431,978, filed on Dec. 9, 2016.

(51) Int. Cl. A61B 5/20; A61F 5/44

(52) U.S. Cl. CPC A61B 5/207; A61B 5/201; A61M 25/0017; A61M 2205/18; A61F 5/4404

(57) ABSTRACT

Fluid monitoring devices and/or systems are provided for monitoring fluid output, including volume and flow rate. A high resolution, low cost electronic fluid monitoring device and system collects fluid such as urine and includes a force sensor that correlates the force of gravity on a fluid collection container with strain and converts it to electrical energy. The force registered on the sensor is correlated with fluid content using a pre-programmed machine learning based algorithm and can be used to identify fluid volume and flow rate. Vertical adjustment and rotational adjustment systems for the device are also described.

### CROSS REFERENCE

This application is a continuation of International Application No. PCT/2017/020981, filed on Mar. 6, 2017, which claims priority to U.S. Provisional Ser. No. 62/303,963, filed Mar. 4, 2016 and U.S. Provisional Ser. No. 62/431,978 filed Dec. 9, 2016, the contents of each of which are incorporated herein by reference thereto.

### FIELD OF THE DISCLOSED SUBJECT MATTER

The present disclosure relates to a detachable, portable fob configured to accurately measure fluid output of objects subject to frequent movement or noise. More particularly, the subject matter pertains to a fluid monitoring system and methods for determining the fluid flow and output from a patient.

### BACKGROUND OF THE DISCLOSED SUBJECT MATTER

There are millions of patients in the United States each year that have an indication for accurate assessment of body fluid output, including urine, pericardial fluid via a pericardial drain, or pleural fluid via a chest tube (heart failure, sepsis, at risk for kidney failure, cardiac tamponade, post-operative, etc.) to name a few. Accurate assessment of the output of these exemplary fluids is important. As an example, accurate assessment of urine output is critical for patient management in both the inpatient and outpatient settings. Urine output measurement is an important part of patient monitoring and is used by clinicians to assess the efficacy of their treatment strategies and can alert clinicians to major problems that may not be caught early enough on physical exam. Urinary drainage containers or bags are conventionally used in hospitals and health care facilities when it is necessary to collect urine from a catheterized patient over a period of time. These containers/bags permit the patient to remain in bed without having to be moved to use a bathroom or a bedpan. Urinary drainage systems may include a catheter (e.g., a Foley catheter), a collection container/bag (e.g., a bag made of a polymeric material or PVC film), and tubing connecting the Foley catheter to the collection container/bag. In operation, the patient is first catheterized, and the catheter is then connected to the drainage container/bag through a length of tubing. The urine drains through the catheter, the tubing, and then finally into the collection container/bag. The urine may be moved from the catheter into the collection bag solely due to gravitational forces. On average, about 80-90 mL (0.5-1 mL/kg/hour) of urine is produced in 1 hour, although individual patients may produce significantly less or more than that depending on their underlying condition and medication regimens.

In yet another example, a drain is often used to prevent fluid (e.g., blood) build-up in a closed (“dead”) space, which may cause either disruption of the healing process for a wound, or cause hemodynamic compromise or an infected abscess. Either scenario may require formal drainage/repair procedure (and possibly another trip to the operating room). Drainage of fluid from a patient may be accomplished by gravity flow of fluid from an interior body space through tubing such as a catheter to a collection vessel. The accurate measurement and close, regular monitoring of bodily fluids from such drains can be important for patient care. Monitoring pericardial or pleural fluid drainage can signify that a drain is blocked and needs to be reopened or that the fluid collection has been completely drained and the drain can be removed.

Inpatient assessment of fluid output relies on ancillary staff to manually record fluid volumes. In some instances, urine volume is tracked by removing urine collection containers/bags after they are filled and then measuring the volume post-collection, but this fails to track volume and flow rate during urination and can delay detection of problems. Pericardial fluid output is monitored by nurses manually measuring the amount of pericardial fluid that drains into a pericardial drain. Pleural fluid output is monitored by nurses manually measuring the amount of pleural fluid that drains into a pleural drain. Due to the general high workload of ancillary staff inpatient urine output measurements are often documented at irregular intervals and are prone to significant documentation errors. Similarly, documentation of pericardial drain fluid output and pleural drain fluid output requires nurses to manually measure and document these volumes. Outpatient assessment of urine output is practically non-existent and may be limited to surrogate measures such as total body weight.

There remains a need for a device and method to efficiently and accurately measure and record fluid output in patients. Consequently, it is desirable to develop low cost, small, reusable, portable, high resolution fluid monitoring devices and systems for monitoring/measuring fluid volume, flow rate, and other parameters that can be easily and rapidly deployed in a busy clinical setting as needed without requiring much space.

### SUMMARY OF THE DISCLOSED SUBJECT MATTER

In one aspect of the present disclosure is a device for measuring fluid output, comprising a compact housing having top and bottom surfaces and sidewalls defining an enclosed space, and a fastener physically engaged to the compact housing and configured to removably attach the device to another object. The fastener is operatively connected to a measuring mechanism configured to measure the weight or mass of the attached object. The compact housing contains a subsystem comprising electronics, such as a microcontroller or processor, in operative connection with the measuring mechanism. The electronics are capable of recording change in weight of an object to which the device is attached over time. In particular, the device is adapted to measure mass or weight or volume of fluid output expelled from a mammal. In one embodiment, the device is for measuring urine output in a subject wearing a urine collection bag, such as a Foley catheter collection bag. In this embodiment, the fastener (e.g., hook or clip) is engaged to the compact housing described above, and is configured to removably attach the device to the urine collection bag. The weight of the fluid collection container is accurately monitored using a strain gauge load cell. A strain gauge load cell measures weight based on the elastic deformation of the load cell's metal spring element. The electronics contained in the housing are adapted to measure mass of urine in the urine collection bag. In this regard, the electronics include an algorithm for assessing when changes in the recorded mass reflect changes in urine volume. The electronics may be programmed to measure mass of the urine periodically.

In order for the strain gauge load cell to function properly the force generated by the weight of the fluid collection container must be completely supported by the load cell and be directed along the principal axis of the load cell. Lateral forces, non-central load application, non-axial load application, and torsion moments, and weight directed from the fluid collection container into the floor are desirably avoided so that the force registered by the load cell is an accurate reflection of the weight of the fluid collection container and the load cell is not damaged.

It has been surprisingly found that accurate measurement of fluid output is improved when the device is capable of: 1) maintaining the orientation of the principal axis of the load cell directed vertically; and 2) maintaining the fluid collection container suspended freely from the load cell completely above the floor. Therefore, the device is also physically engaged to an adjustable, rotational support and an adjustable vertical mechanical support. In some embodiments, the rotational support and the vertical support may be separate systems. In other embodiments, the rotational support and the vertical mechanical support comprise a combined support system. In preferred embodiments, the rotational support and the vertical support are operatively connected to a microcontroller that is configured to implement a mechanical algorithm to adjust the rotational support and the vertical support to allow for accurate measurement of the fluid.

The device may further include a display. The display may be integral with the compact housing or a second device. The display has an output for information relating to the fluid output. For example but not limitation, the information may include mass of the object to which the device is attached, such as a urine collection container, pericardial fluid collection container, pleural fluid collection container, or handheld urinal.

The housing subsystem may further include memory for storing measurements made by the processor. A transceiver may be included for sending information to and receiving information from a remote location. In one embodiment, the transceiver is capable of sending measurement information assessed by the microcontroller to a remote location. Thus, the device may be part of a larger integrated diuresis management program or service. For example, the device may be part of a system, such as a physician management system or administration review system or an alert system. For example, the device can be configured to provide alerts of low or high urine output from an individual patient.

In another aspect, the device is a handheld, portable device that can be easily attached to any standard hospital bed (or compatible surface) and continuously monitor body fluid output. The device has the capability of filtering out noise that occurs as a result of the busy clinical environment including: 1) a nurse emptying the collection container; 2) inadvertent disturbance of the collection container by clinical staff or patient; 3) transporting of a patient for a test or procedure, and removal of the fluid collection container from the device; 4) the bed being adjusted too low to the floor so that the fluid collection container is touching the floor thereby causing the force from the container to be directed into the floor rather than the load cell; 5) the bed being adjusted at an angle so that the weight of the collection container is no longer directed along the principal axis of the load cell thereby causing the force from the collection container to not be registered accurately by the load cell.

In some embodiments, the device is part of a larger system that has the capability of displaying data in various formats including: 1) a touch screen display on the device; 2) via an online web portal; 3) via direct integration into the electronic medical record. These capabilities make this device ideally suited for monitoring fluid output in patients in a variety of settings including in a hospital, at home, or in rehabilitation center.

In one embodiment, the device comprises: a load cell; electronics, such as a microcontroller; a touch screen; WiFi shield; gyroscope; mechanical elevation system; mechanical rotational system; force monitoring system; clamp; and optionally, shielding to protect the load bar from external objects.

In accordance with one embodiment during operation, the device is attached to a hospital bed (or compatible surface) and turned on. The device may connect to the local WiFi network. The device displays either a randomly generated code or the user can input an identifier that identifies that fluid output monitoring session. The fluid collection container is placed on a load cell integrally attached to the device housing, such as by use of a hook. The device may further include a shield to protect the load bar from external objects (e.g. blankets, pillows, body parts, etc.). The device sets the start point at zero ml. Every few seconds or minutes or hours, depending on the time period chosen, the device electronics measure and report or display fluid output. A pre-programmed, machine learning based algorithm is used to filter out noise. The algorithm may use running average and Gaussian filter with pre-specified cutoffs to distinguish noise from an accurate measurement. When the fluid collection container is removed from the device the weight will drop below a pre-specified threshold and the device will wait until the container is replaced before taking another measurement. When the container is replaced, if the electronics assess that the new average is higher than the previous measurement, the difference will be added as new urine output measurement. If the measurement is less than the previous measurement, the difference will be ignored and the volume monitoring will continue. The device further includes a level determining device (e.g. gyroscope) that monitors the geometric position of the device relative to the floor. If the gyroscope senses that the device and/or its attached load cell is not oriented such that the principal axis of the load cell is not substantially directed vertically (such as less than 15 degrees, less than 10 degrees, or preferably less than 5 degrees from vertical) then the microcontroller sends a message to the mechanical rotation support to rotate the device housing so that it orients the principal axis of the attached load cell vertically. The gyroscope is built into the housing and the processor that regulates the rotational system may be the same processor that regulates the data collection from the load cell and algorithm, or it may be a separate processor in the microcontroller.

The fluid output data is displayed in a convenient table format with new and cumulative fluid outputs listed periodically, such as hourly. The fluid output data can be sent to an online server linked only to the randomly generated code on the device. No protected health information needs to be sent for this. The fluid output data can also be sent from the device using a user-entered identifier, which may include protected health information. The data can also be generated in a format that can be incorporated into an electronic medical record.

The device described and embodied herein may have many applications. For example but not limitation, the device may be used for assessing response to diuretic therapy in patients with heart failure, early diagnosis of acute kidney injury in patients, monitoring drainage of pericardial fluid, or monitoring drainage of pleural fluid.

The device may be compatible with collection containers that continuously monitor fluid output or handheld urinals that are only placed on the device intermittently. The device uses an algorithm to change the mechanical position of the device in order to optimize accuracy. The device uses an algorithm to take into account patient being removed for a test and then coming back. The device can output data in a format that can be incorporated into the EMR. The device is designed with the capability of functioning without needing to store or transfer any protected health information so there would be no issue with HIPAA while being able to provide clinical data.

In some embodiments, a fluid measuring or monitoring system is provided that comprises a container for collecting a fluid such as urine, pericardial fluid or pleural fluid; a force-sensing assembly configured to sense a physical property of the fluid as it collects in the container and provide a measurement value indicative of volume of the fluid as it collects in the container, and a processor programmed to calculate a volume measurement of the fluid as it collects in the container based on data received from the sensor using a pre-programmed, machine learning based algorithm. Preferably, the physical property of the fluid is its weight.

For example, the fluid monitoring device may comprise (a) a support and measurement assembly comprising a support member configured to support a container for collecting fluid and a sensor in operational communication with the support member wherein the sensor is configured to measure the weight of the container and fluid collected therein and provide an electrical signal proportional to the measured weight; and (b) a microcontroller in electrical connection with the sensor, configured to signal the sensor to weigh the container periodically, receive the electrical signal from the sensor and to use data from the sensor to calculate a volume of the fluid collected in the container using a pre-programmed machine learning based algorithm.

In some embodiments, the support and measurement assembly comprises a strain gauge load cell to measure the weight and provide the electrical signal. The load cell may comprise a bending beam load cell or tension load cell capable of translating up to 10 kg of force with an accuracy of 1-2 g into an electrical signal.

The microcontroller may include software programmed to analyze the raw weight of the fluid collection container and then convert this data into a volume using the machine learning based algorithm. The algorithm may use at least one of a linearization procedure or a curve fitting procedure. The algorithm includes a decision tree format. The algorithm converts the changing weight of the container into a volume while filtering out signal noise from the clinical environment. The algorithm assesses the validity of each data point.

In some embodiments, the algorithm comprises the parameters of:
1) repeating measurements at regular intervals;
2) averaging a series of measurements to provide a running average;
3) determining that the running average meets a minimum value cut off and a minimum standard deviation cutoff in order to be accepted;
4) comparing the new accepted average to a previously accepted average;
5) determining whether the device adds new data to the cumulative or restarts from a new baseline based on whether the new accepted average is higher or lower than the previous accepted average; and
6) adjusting the cutoff values based on analysis of current fluid output flow rates.

The algorithm uses a running average, standard deviation and comparison to previously accepted values to determine fluid output.

The algorithm wherein the (1) the running average and a standard deviation are calculated based on 3 to 10 measurements with measurement intervals from 10 seconds to 60 seconds, and wherein (a) if the running average is less than a threshold from 20 g to 100 g, then all measurements are rejected; (b) if the running average is greater than the threshold from 20 g to 100 g and the standard deviation is greater than a threshold from 3 to 15, then all measurements are rejected; or (c) if the running average is greater than the threshold from 20 g to 100 g and the standard deviation is less than the threshold from 3 to 15, the average weight is accepted; (2) the accepted average weight is subtracted from a previous accepted average weight to provide a new interval weight; wherein (a) if the new interval weight is positive, then the new interval weight is added to the cumulative weight; or (b) if the new interval weight is negative, then the device records the new interval weight as a new zero and the cumulative weight is unchanged.

The microcontroller may include software programmed to analyze the position of the device in space as assessed by a level determining device and control a rotational motor system to reposition the device so that the principal axis of the strain gauge load cell remains directed vertically.

The microcontroller may include software programmed to analyze the height of the device from the floor and control a vertical motor system to increase the height of the load cell if the fluid collection container is too close to the floor.

The fluid monitoring device may further comprise a wireless transceiver for transmitting the volume measurement to a separate device.

The fluid monitoring device wherein the microcontroller includes software programmed to transmit the volume measurement with a unique identifier to distinguish the volume transmitted by the fluid monitoring system from data transmitted by other monitoring systems.

The fluid monitoring device wherein the microcontroller includes software programmed to transmit the volume measurement with a programming language compatible with an electronic medical record system.

The fluid monitoring device further comprises a display screen that can display information.

The fluid monitoring device wherein the information is selected from the group consisting of measured fluid volume, flow rate, function code, identification code, instructions, and any combination thereof.

The fluid monitoring device further comprises a touch screen configured for inputting information into the device.

The fluid monitoring device further comprises an attachment member configured to attach the device to a supporting apparatus.

The fluid monitoring device wherein the attachment member comprises at least one clamp, hook, bracket or strap.

The fluid monitoring device wherein the device includes a mechanism for implementing a mechanical algorithm for positioning the device vertically so that 1) the device is mounted sufficiently high enough so that the collection container can hang freely from the support member without touching any surface that would prevent the device from measuring the weight of the fluid collection container and fluid therein; and 2) the device is adjusted rotationally so that the principal axis of the load cell is directed vertically.

The fluid monitoring device wherein the mechanism to position the device vertically comprises a bracket slidably engaged to a track configured to allow the device to move vertically along the track, or a vertical rack and pinion system.

The fluid monitoring device wherein the device comprises at least one level-determining device selected from the group consisting of a gyroscope, bull's eye spirit level, inclinometer, electronic tilt sensor, accelerometer, liquid capacitive level, electrolytic level, gas bubble in liquid level, pendulum level, and micro-electro-mechanical system level.

The fluid monitoring device wherein the mechanism to adjust the device rotationally comprises a screw, shim, gear or axle.

Also provided is fluid monitoring system, comprising a device according to any of above embodiments; and (c) a container for collecting a fluid.

Embodiments of the system include the following:

The fluid monitoring system wherein the container is a urine collection container, pericardial fluid collection container, pleural fluid collection container, or a handheld urinal.

The fluid monitoring system wherein the container is operationally connected to a fluid collecting device selected from the group consisting of a urine catheter, pericardial drain, chest tube, and Jackson-Pratt drain.

The fluid monitoring system wherein the fluid measured is urine, pericardial fluid, pleural fluid, blood, or blood serum.

The fluid monitoring system wherein data obtained by the device is displayed on the display screen of the device, or transmitted via Wi-Fi over the internet to a dedicated local server or a cloud based system and accessed from a secure website, or directly integrated into an electronic medical record system.

Also provided is a method of measuring a fluid volume, comprising: providing a fluid measuring device according to any of the above embodiments; contacting a fluid collection container with the fluid measuring device in a manner configured to enable weighing of the container and fluid collected therein; collecting a fluid in the fluid collection container; measuring the weight of the fluid collection container and the fluid collected therein and converting the weight measurement to an electrical signal proportional to the weight using the sensor; sending the electrical signal to the microcontroller; and calculating a volume of the fluid as it collects in the container based on data from the sensor.

Embodiments of the method include the following:

The method further comprising implementing a mechanical algorithm for positioning the device vertically so that 1) the device is mounted sufficiently high enough so that the collection container can hang freely from the support member without touching any surface that would prevent the device from measuring the weight of the fluid collection container and fluid therein; and 2) the device is adjusted rotationally to be in a configuration wherein the principal axis of the load cell is directed vertically and maintains the weight of the fluid collection container aligned with the principal axis.

The method wherein calculating the volume of the fluid comprises using the machine learning based algorithm.

The method wherein the algorithm uses at least one of a linearization procedure or a curve fitting procedure.

The method wherein the algorithm includes a decision tree format.

The method wherein the algorithm converts the changing weight of the container into a volume while filtering out signal noise from the clinical environment.

The method wherein the algorithm assesses the validity of each data point.

The method wherein the algorithm comprises the parameters of:
1) repeating measurements at regular intervals;
2) averaging a series of measurements to provide a running average;
3) determining that the running average meets a minimum value cut off and a minimum standard deviation cutoff in order to be accepted;
4) comparing the new accepted average to a previously accepted average;
5) determining whether the device adds new data to the cumulative or restarts from a new baseline based on whether the new accepted average is higher or lower than the previous accepted average; and
6) adjusting the cutoff values based on analysis of current fluid output flow rates.

The method wherein the algorithm uses a running average, standard deviation and comparison to previously accepted values to determine fluid output.

The method wherein the (1) the running average and a standard deviation are calculated based on 3 to 10 measurements with measurement intervals from 10 seconds to 60 seconds, and wherein (a) if the running average is less than a threshold from 20 g to 100 g, then all measurements are rejected; (b) if the running average is greater than the threshold from 20 g to 100 g and the standard deviation is greater than a threshold from 3 to 15, then all measurements are rejected; or (c) if the running average is greater than the threshold from 20 g to 100 g and the standard deviation is less than the threshold from 3 to 15, the average weight is accepted; (2) the accepted average weight is subtracted from a previous accepted average weight to provide a new interval weight; wherein (a) if the new interval weight is positive, then the new interval weight is added to the cumulative weight; or (b) if the new interval weight is negative, then the device records the new interval weight as a new zero and the cumulative weight is unchanged.

The method wherein the container is a urine collection container, pericardial fluid collection container, pleural fluid collection container, or handheld urinal.

The method wherein the container is operationally connected to a fluid collecting device selected from the group consisting of a urine catheter, pericardial drain, chest tube, and Jackson-Pratt drain.

The method wherein the fluid measured is urine, pericardial fluid, pleural fluid, blood, or blood serum.

The method wherein data obtained by the device is displayed on the display screen of the device, or transmitted by Wi-Fi over the interne to a dedicated local server or a cloud based system and accessed from a secure website, or directly integrated into an electronic medical record system.

The method further comprising calculating a flow rate of the fluid as it collects in the container based on data from the sensor.

In one aspect of the present disclosure is a device for measuring fluid output, comprising a compact housing defining an enclosed space, the compact housing shaped as a fob, and a fastener engaged to the compact housing and configured to removably attach the device to another object, wherein the compact housing contains a processor adapted to measure mass of an object to which the device is attached. The processor is capable of recording change in weight of an object to which it is attached over time. In one embodiment, the device is for measuring urine output in a subject wearing a urine collection bag, such as a Foley catheter collection bag. In this embodiment, the fastener (e.g., hook or clip) is engaged to the compact housing described above, and is configured to removably attach the device to the urine collection bag. The electronics contained in the fob are adapted to measure mass of urine in the urine collection bag. In this regard, the electronics include an algorithm for assessing when changes in the recorded mass reflect changes in urine volume. Accordingly, more accurate measurements are achieved. The electronics may be programmed to measure mass of the urine periodically. In a second embodiment, the device is for measuring urine output in a subject holding a handheld urinal whereby the subject hangs the urinal from the device after filling the container.

An embodiment comprises a portable reusable device for measuring fluid output from a patient, comprising a housing including a top and bottom surface and sidewalls defining an enclosed space, the housing including a subsystem including a processor configured to measure fluid output expelled from a patient, a fastener to removably attach the device to an object, and an adjustable, rotational mechanical support physically engaged to the housing and operatively engaged to a gyroscope, wherein the gyroscope monitors the device position and further wherein the mechanical support rotates the device position upon feedback related to the gyroscope monitoring.

The device may further include a display. The display may be integral with the compact housing or a second device. The display has an output for information relating to the mass of the object to which the device is attached. The information includes urine output and the object is a urine collection bag or handheld urinal. The information may also include data to determine diuretic dosing information, patient weight information, BUN/Cr information, or potassium ion information.

The compact housing further includes a memory for storing measurements made by the processor. A transceiver may be included for sending information to and receiving information from a remote location. In one embodiment, the transceiver is capable of sending measurement information assessed by the processor to a remote location. Thus, the device may be part of a larger integrated diuresis management program or service. For example, the device may be part of a system, such as a physician management system or administration review system or an alert system. For example, the device can be configured to provide alerts of low or high urine output from an individual patient.

### BRIEF DESCRIPTION OF THE DRAWINGS

FIG. 1 shows a drawing of an exploded view of a specific embodiment of the device as described herein.

FIG. 2 shows drawings of top, side and perspective views of the assembled device of FIG. 1.

FIG. 3 shows a photograph of an embodiment of the fluid measurement system in a hospital setting.

FIG. 4 shows a schematic drawing of a device with an alternative attachment member including a rotational mechanical system and a vertical mechanical system.

FIGS. 5A and 5B show schematic views of the device attached to a hospital bed in various configurations.

FIGS. 6A to 6C show schematic views of the device attached to a hospital bed in various configurations.

FIGS. 7A to 7C show schematic views of an alternative embodiment of the device.

FIG. 8 shows a decision tree, machine learning approach to the algorithm used in the device.

FIG. 9 shows a comparison of raw data and corrected data for measuring a fluid.

FIGS. 10A to 10D show comparisons of measurements by the device to measurements by trained clinicians.

Component identifier numbers are the same for like components in each of the Figures.

### DETAILED DESCRIPTION OF THE DISCLOSED SUBJECT MATTER

As used herein, the words “including,” “has,” and “having,” as used herein, including the claims, shall have the same meaning as the word “comprising.”

The term “accuracy” refers to a measure of rightness, e.g., the agreement between a measurement and the true or correct value. While accuracy refers to the agreement of the measurement and the true value, it does not tell you about the quality of the instrument used. “Error” refers to the disagreement between a measurement and the true or accepted value. “Precision” is a measure of exactness and refers to the repeatability of measurement. “Resolution” refers to the minimal change of the input necessary to produce a detectable change at the output. “Transducer” refers to a device that transfers energy between two systems such as the conversion of mechanical forces into electrical energy.

As used herein, the term “handheld” refers to an object that can be held in or by a single adult human hand. The term does not require that the object be held in or by the hand during its use.

A “processor” is the logic circuitry that responds to and processes the basic instructions that drive a computer. The term “microcontroller” refers to a small computer on a single integrated circuit. A microcontroller contains one or more processor cores along with memory and programmable input/output peripherals. It may also be linked to and/or control other components, such as displays and/or actuators in the device, and/or remote displays, stations, databases, and the like. The term “electronics” as used herein refers to any electrically powered or controlled component of the device. These terms may be used interchangeably herein when referring to operations of the device, such as for calculations, operating algorithms, or controlling motors, servos and/or actuators.

A “load cell” is a device that converts a force or load into a measureable output. Strain gauge load cells convert a force or load into an equivalent electrical signal or digitized load value. “Strain gauges” are resistors that change their resistance proportional to load as they are deformed. They consist of a pattern of resistive foil mounted on a backing material. When bonded to an object (see “spring element” below) they will deform as the object does when a load is applied. As the strain gauge stretches, its resistance increases, and as it compresses, its resistance decreases. The amount of change in resistance indicates the magnitude of deformation.

The “principal axis” (or primary axis or z-axis) of a load cell is the axis along which the load cell is designed to be loaded. An “axial load” is a load applied along the principal axis of the load cell, allowing for the most accurate measurement. An “inclined load” is a load applied at an angle to the principal axis and measurement of the load will be less accurate and the load cell may be damaged. For the devices described herein, the load is the weight (downward force) of the fluid collection container and the principal axis of the load cell should be directed vertically for accurate measurement of the weight.

A critical mechanical component in any strain gauge based sensor is the “spring element.” In general terms, the spring element serves as the reaction to the applied force, load, or weight and focuses it into a uniform, calculated strain path for precise measurement by the bonded strain gauge. In a “bending beam load cell”, the principal axis is at right angles to the beam spring element and the applied load is measured based on the amount of deflection or bending of the beam. In a “column” or “tension load cell”, the principal axis is aligned with the spring element and the applied load is measured based on the amount of stretching of the spring element.

Provided herein are fluid/urine monitoring devices and systems including features believed to provide advantages over existing fluid/urine meters. The portable, small, reliable, reusable, low cost fluid (e.g., urine) monitoring devices and/or systems of this disclosure include weight-based transducer measurement systems (e.g., load cell or strain gauge systems).

Although the device, systems and methods are described herein primarily with reference to collection and measurement of urine, the device, systems and methods are not limited to urine measurement. They are also suitable for measuring other bodily fluids, including pericardial fluid, pleural fluid, blood, and the like.

There are millions of patients in the United States each year that have an indication for accurate assessment of urine output (heart failure, sepsis, at risk for kidney failure, etc.). Inpatient assessment of urine output relies on ancillary staff to manually record urine volumes from indwelling urine catheter bags or handheld urinals. Outpatient assessment of urine output is practically non-existent and limited to surrogate measures such as total body weight.

These circumstances result in problems in patient assessment, including:

1) Due to the general high workload of ancillary staff inpatient urine output measurements are often documented at irregular intervals and are prone to significant documentation errors. The current system for monitoring patient urine output is for nurses to manually measure the amount of urine contained in a urine collection container and then record the data into the electronic medical record (EMR). This system is cumbersome, inefficient, and time consuming. It is also prone to errors, including inaccurate assessment of urine output and/or transcription errors. Therefore, critical medical management decisions (e.g. diuretic regimens, nephrotoxic medications) may be made without optimal data resulting in prolonged patient length-of-stay; and

2) Information on outpatient urine output is not readily available for outpatient physicians, making it difficult for them to manage their patients. This may lead to an increase in hospitalizations. Furthermore, patients in nursing homes with indwelling catheters may have decreased urine output which is a sign of infection or dehydration that is often missed due to poor monitoring, resulting in increased hospitalizations.

The handheld, portable system for monitoring body fluid output described herein can be easily adapted into current clinical practice for use with inpatients and outpatients including patients in hospitals and rehab facilities. It employs a hanging scale with a pre-programmed algorithm and wireless capabilities that can be easily attached to urine (or other fluid) collection devices that are currently used in every hospital.

Described herein is a small, handheld, portable, reusable device that can attach to a hospital bed and includes an electronic scale (such as in the form of a load cell or strain gauge) for weighing a standard commercially available fluid collection container (indwelling urinary catheter bag, handheld urinal, pericardial drain, pleural fluid drain, etc.) and is operated by:

1) implementing a mechanical algorithm that adjusts the position of the load cell in order to maintain it high enough so that the fluid collection container can hang freely from the device and in a configuration that constantly maintains the weight of the fluid collection container aligned with the principal axis of the load cell; and

2) implementing a software algorithm to convert the changing weight of the container into a volume while filtering out signal noise from the clinical environment; and

The raw data is analyzed by a processor within the device microcontroller which applies the algorithm to assess the validity of each data point.

The device also desirably contains a display screen (such as an LCD screen) that can display information such as fluid volume and/or flow, function codes, and identification numbers.

Fluid measurement data obtained by the device can be output and accessed by a clinician in one or more of three ways:
1) Data (i.e. hourly fluid output, cumulative output, etc . . . ) can be displayed on the display screen of the device, which nurses can refer to and then manually input the data into the hospital EMR.
2) Data can be transmitted via Wi-Fi over the interne to a dedicated local server or a cloud based system and accessed from a secure website, with data stored on dedicated servers or in data clouds. The device is designed so that the data sent over wireless Wi-Fi to the server does not need to include protected health information, which reduces privacy security risks. Authorized users can log in to view fluid output data using a secure web-browser. Physicians can refer directly to this information or else nurses can view the data and manually input it into the hospital EMR.
3) Data can be directly integrated into EMR systems using appropriate software/drivers stored locally on the facility's server(s).

These three levels of data storage and retrieval make it easy for an institution to use the device to measure, store and access fluid measurement data.

This device would provide physicians with live, accurate urine output measurements that could accelerate medical decision making. This system would make it possible to optimize management strategies for shock, heart failure and sepsis and for early detection of acute kidney injury. For example, if a physician administers a diuretic medication such as furosemide, the device provides immediate access to the patient's urine output response to the medication over the next six hours. Based on this data the physician can decide to re-administer a higher dose, add a new medication or wait. This could expedite time to discharge or consideration for escalation of therapy. Furthermore, automating urine output measurement and charting will significantly reduce nursing staff workload.

This device would also make it possible to easily monitor pericardial or pleural fluid drainage so that physicians would be aware when the rate of drainage changes. This can signify that a drain is blocked and needs to be reopened or that the fluid to be collected has been completely drained and the drain can be removed.

The physician may be able to better manage patient care by allowing for optimal diuretic dosing based on urine output response to a specific medication, dose or formulation. The urine output data is readily available for physicians to use for medically managing patients.

The system described herein will help physicians to manage heart failure, sepsis, acute kidney injury and post-operative drains in real time, which could allow frequent adjustments to medication regimens throughout the day. This has the potential to reduce patient length-of-stay. It will also give physicians the ability to closely monitor their patients' urine output in a rehabilitation facility or at home so that medication doses (e.g. diuretics) can be effectively titrated as an outpatient. This system can be used in long-term care facilities to monitor patients' urine output. A precipitous decrease in urine output can signal early development of sepsis or acute kidney injury enabling outpatient physicians to quickly start antibiotics or intravenous fluids thereby preventing the need to transfer the patient to a hospital for higher level of care. In the outpatient and rehab settings physicians could use this information to effectively titrate outpatient medication regimens or detect infections or dehydration earlier preventing costly hospital admissions for decompensated heart failure, acute kidney injury, and sepsis. Thus, this disclosed subject matter has the potential to reduce hospital admissions.

The following description and accompanying figures, which describe and show certain embodiments, are made to demonstrate, in a non-limiting manner, a small, reliable, low cost fluid (e.g., urine) monitoring apparatus and/or system, including for measuring volume and flow rate, according to various aspects and features of the present disclosure. The devices and systems disclosed may be used as urine monitoring devices/systems, or may be used to monitor other fluids in various applications.

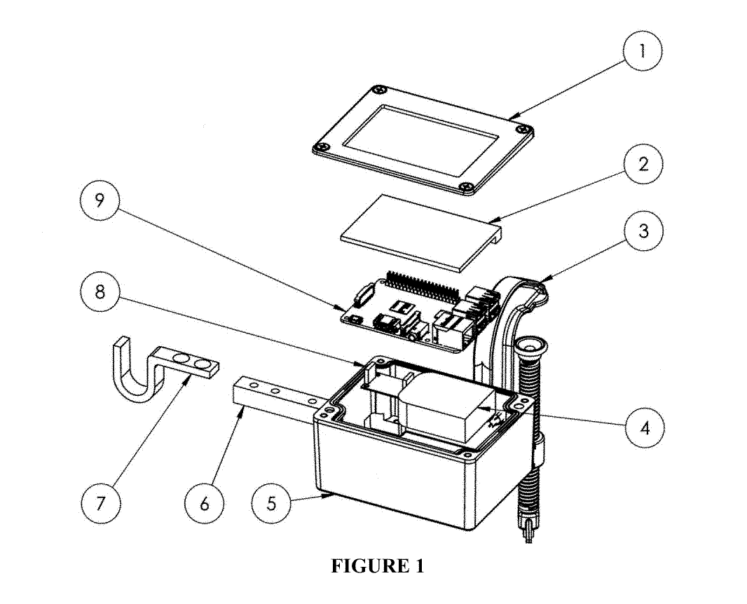

The fluid measuring device and system may be understood with reference to FIGS. 1-7.

The fluid measuring device comprises

(a) a support and measurement assembly comprising a support member configured to support a container for collecting fluid and a sensor in operational communication with the support wherein the sensor is configured to measure the weight of the container and fluid collected therein and provide an electrical signal proportional to the measured weight; and

(b) a microcontroller in electrical connection with the sensor, configured to signal the sensor to weigh the container periodically, receive electrical signals from the sensor and to use data from the sensor to calculate a volume of the fluid as it collects in the container.

In one embodiment, the fluid container may be arranged such that it hangs from a support and measurement assembly. The support and measurement assembly may comprise a hook or bracket configured to support a free-hanging collection container, such as a standard fluid collection bag or a handheld disposable urinal. The support is in operational communication with a force sensor such as a strain gauge apparatus that is configured to measure the strain induced by the weight of the collection bag on the support. As the fluid container fills with fluid (e.g., urine), its weight increases and it pulls more heavily downward on the support, which in turn provides more force to the force sensor strain gauge. In this way, the support and measurement assembly of the urine measurement device can be used with any size or type of fluid container to measure the increase in weight or downward force as the fluid container fills with fluid. This increase in downward force or weight can be correlated to volume increase to give a measurement of volume and flow rate.

In a specific embodiment as shown in FIG. 1 in an exploded view, the support and measurement assembly comprises a support member such as a bracket or hook 7 configured to suspend a free hanging fluid collection container (not shown in FIG. 1; 10 in FIG. 3). Other arrangement designs in keeping with the principles described herein are also contemplated. Alternative support members 7 may comprise a rigid pan on which a fluid collection container is placed; or a fabric or mesh netting that supports the fluid collection container, either by suspending the container below the device or supporting the container as in a basket.

In any configuration, the support member is in operational communication with a sensor 6 that is configured to measure the weight of the fluid container and fluid such as urine collected therein. Preferably the sensor 6 is a strain gauge load cell that measures the downward force caused by the fluid collection container held by the support member and converts the observed force into an electrical signal proportional to the weight of the fluid collection container and collected fluid.

The fluid measuring device desirably includes a load cell 6, such as a bending beam load cell. A specific embodiment comprises a bending beam (bar) load cell (80 mm×12.7 mm×12.7 mm) that can translate up to 10 kg of pressure (force) with an accuracy of 1-2 g into an electrical signal. Each load cell is able to measure the electrical resistance that changes in response to, and proportional with, the strain (e.g. pressure or force) applied to the bar. The load cell is used to measure the weight of the suspended fluid collection bag and whether the bag's weight changes over time by measuring the strain or load applied to the gauge. The gauge may be made of any suitable material, such as an aluminum-alloy. In bending beam load cells, the cell is set up in a “Z” formation so that torque is applied to the bar and the four strain gauges on the cell will measure the bending distortion, two measuring compression and two tension. In the device herein, the bar is oriented horizontally and weight (force) is applied vertically to one end of the bar and aligned with the principal axis of the load cell. Strain is determined based on the deflection of the bar. In one embodiment, the load cell has four strain gauges that are hooked up in a Wheatstone bridge formation. When these four strain gauges are set up in a Wheatstone bridge formation, it is easy to accurately measure the small changes in resistance from the strain gauges. The load cell may also comprise wiring to connect to the microcontroller described below and attachment components for attaching to the collector support hook 7, such as two M4 and two M5 sized through holes for mounting purposes.

The fluid measuring device desirably includes a load cell amplifier 8, a small breakout board electrically connecting the load cell to the microcontroller that allows reading of the changes in the resistance of the load cell to measure weight. After calibration the system is capable of providing very accurate weight measurements. A specific embodiment comprises separated analog and digital supply, a 3.3 uH inductor and a 0.1 uF filter capacitor for digital supply.

The device also comprises a microcontroller 9. In the microcontroller, the measurement data from the sensor and data from the other features discussed herein are processed by written software or firmware. This software/firmware consists of functions which combine the measurements data to produce usable quantities for the user, and will be described in more detail below. In one embodiment, the microcontroller may be a 64-bit microcontroller. A specific example is a Raspberry Pi 3 Model B comprising a Quad Core Broadcom BCM2837 64-bit ARMv8 processor, with processor speed up to 1.2 GHz. The microcontroller board provides a complete, high-quality development platform for series devices. It may include a variety of on-board modules (Ethernet PHY), I2C, SPI, RTC, audio codec, accelerometer, temperature sensor, and flash memory, which allows writing applications of high complexity quicker. This microcontroller platform also comprises a built-in BCM43143 WiFi chip and Bluetooth Low Energy (BLE) capability for wireless communication to other devices.

The device also desirably can generate and display a new randomly generated device identification number each time that the device is turned on. The number is used to identify the device for data transfer to a remote location, such as an interne based server. This provides a system for transmitting data without having to transmit protected health information. As an illustration, when the device is turned on a code is generated by the device that may be displayed on the device. This code is sent to the server where a file is generated consisting of readings from that device. The patient's name and medical record number is linked to that device on the server. This allows data to be transmitted from the device to the server that is free of protected health information. This makes it possible for the device to store and send de-identified patient information. It is the responsibility of the party collecting the data to know which identification number corresponds to a specific patient.

The data is sent to a server using wireless communication networks (Wi-Fi) that can be accessed on a HIPAA compliant website. The data can also be synchronized with the patient's electronic medical records by generating data in a compatible format such as POCT1-A compliant HL7 result interface. In one embodiment, the device does not send protected health information, rather it sends data associated with the randomly generated identification number on the device. The patient's name and medical record number is linked to the data generated by the device on the HIPAA protected website.

The fluid measuring system may also include a display or monitor that is programmed to display volume, flow rate, temperature, and/or other parameters based on sensor measurements. The display or monitor may be disposed in a location remote from the measuring device, and/or preferably the measuring device may comprise a display that is incorporated into the device. For example, the LCD screen may display the running cumulative volume of the fluid in the collection container on a periodic basis depending on how closely fluid output needs to be monitored. The updated volume output may be displayed in periods ranging from about once every 20 seconds, or once a minute to about once an hour.

Other information displayed may include function or notification codes that indicate that the device is operating properly and/or needs some attention from a practitioner, such as, for example, indicators that the device needs leveling, when the volume collected is approaching the capacity of the collection container, when the flow rate is increasing or decreasing, and/or when the weight of fluid is no longer increasing or even declining, which may indicate a leak or an improperly closed drain valve. The controller is configured to determine which notification code should be displayed, based on the fluid volume determined by the device.

The notification codes may be linked to visual and/or audible signals or alarms that can signal a practitioner to address the condition of the device. For example, the device may provide an audible signal such as a beep or tone if the device determines that the fluid collection vessel is approaching capacity, or that weight/volume of fluid is decreasing.

In another embodiment, the device has a touch screen that can display a digital keypad where a nurse can enter information such as the patient's medical record number that will be used to link the data generated by the device with the patient's chart on the electronic medical record. Other information entered might include the capacity of the fluid collection device and/or the frequency for displaying output information updates.

As shown in FIG. 1, the fluid measuring device desirably includes a video display such as an LCD screen 2 for displaying information such as volume, flow rate, function codes, and/or patient identity codes. A specific embodiment includes a 3.5-inch (9 cm) TFT display with 480×320 16-bit color pixels. In another embodiment, the screen includes a resistive touch overlay for inputting information. This enables a nurse to input the patient's medical record number or other information into the device. The device uses this information to link the fluid output data generated to the patient's chart in the healthcare facility's electronic medical record.

The fluid measuring device preferably includes a power supply to provide electric power to the electronic components. For example, as shown in FIG. 1, the device includes a battery 4. Many battery options may be contemplated. A specific embodiment of the battery is a 2200 mAh battery. Alternative batteries may include standard replaceable and/or rechargeable battery cells such as alkaline or NiCd batteries such as AAA or AA cells. Preferably, the device includes means for recharging the battery from a separate, remote power source such as common alternating current converted to direct current by an appropriate converter. The connector to the recharging power source may include for example but not limitation a USB or other standardized port. Another embodiment of a power supply comprises an 1800 mAh Aluminum Power Bank charger, which is also connected to a power outlet. Alternatively or additionally, the device may be configured to be powered directly by alternating current through a connected or removable power cord.

The device is desirably designed such that all components including the support and measuring assembly, microcontroller, power source, optional video display and other components necessary for the operation of the device are combined with and/or contained in an enclosure or casing to provide a single self-contained unit that is capable of performing all the functions necessary to measure fluid collection and display and/or transmit the volume information. As shown in FIG. 1, an embodiment of the enclosure or casing includes an enclosure lid 1 and enclosure body 5. Preferably, the walls of the enclosure body 5 and lid 1 are rigid. Lid 1 of this embodiment includes an opening optionally comprising a transparent, flexible window that protects the LCD screen 2 while allowing a user to view the information and interact with the device with the touch screen features. The lid 1 and body 5 are held together by any suitable means to enclose the components of the device. In the embodiment shown in FIG. 1, the lid 1 is secured to the body 5 using 4 screws. The body 5 is configured with various attachment and/or support members to hold the components of the device in their desired positions within or on the casing. The load cell 6 is positioned in the body 5 so that the end in electrical connection with the microcontroller is inside the casing, while the other end extends outside the casing to operationally engage with the support member (hook 7). One skilled in the art can appreciate that the specific design of the casing may have many options depending on the shapes and dimensions of the various components used in the device.

The casing of the device also comprises a member to attach or connect the device to a supporting apparatus that provides a stable location for the device while allowing the device to engage and support the fluid collection container during the collecting and weighing process. The supporting apparatus may include for example, a patient bed or a shelf, stand, cart, table, or the like in proximity to the patient. The attachment member 3 may include clamp(s), hook(s), bracket(s), strap(s), etc. to support or suspend the device near the patient and allow a clinician easy access to the device, the fluid collection container and associated fluid conduits used to collect the fluid from the patient. As shown in FIG. 1, the attachment member comprises a five-inch C-clamp 3 with a screw tightening mechanism on the exterior of the enclosure body 5 configured to hold the device onto a bed rail or other horizontal member near the patient.

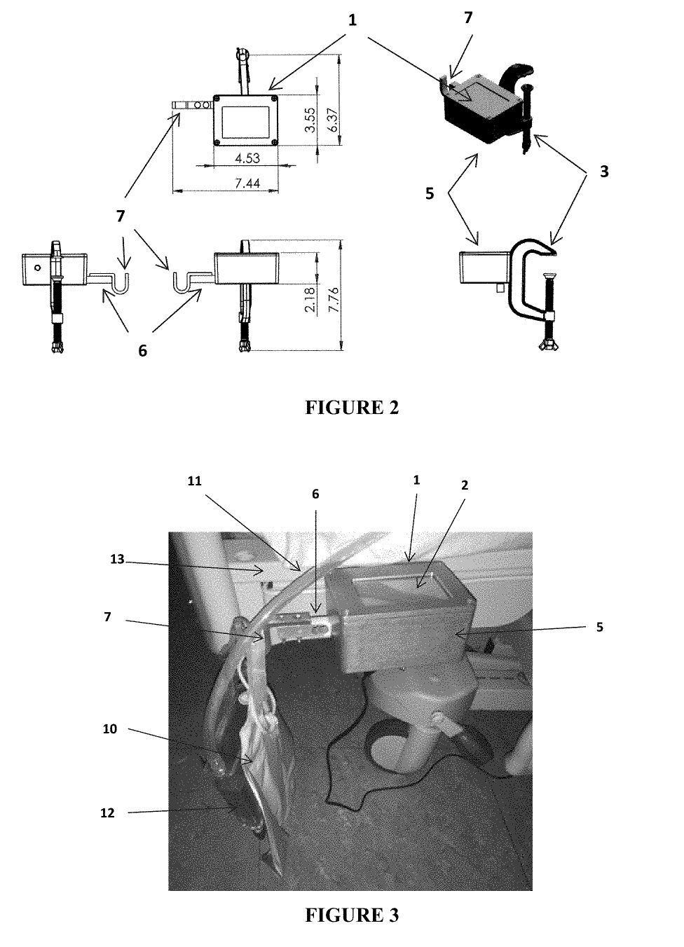

FIG. 2 shows side, top and perspective views drawings of the assembled device illustrated in FIG. 1. The dimensions of the device are shown in inches.

FIG. 3 shows a photograph of a similar embodiment of the fluid measuring device as part of the fluid measuring system in a hospital setting. In this embodiment, the enclosure body 5 is clamped to a bed rail using a clamp (not visible). The load cell 6 is at the bottom of the enclosure body 5 and in operational connectivity to a hook 7. A fluid collection container 10 is suspended from the hook 7. Tubing 11 is configured to convey urine 12 from a patient (not shown) and collected in the container 10.

Importantly, the device described herein does not require a proprietary urinary collection catheter or container to function. The device can work with any fluid collection system, for example, standard indwelling urine collection catheters and containers, provided the fluid container can be hung from the device.

A wide variety of types of fluid collection containers 10 or bags may be used for the fluid collection container. For example, the container 10 may be, or similar to, any known fluid collection container or fluid collection bag. The container/bag may take a variety of sizes, shapes, and forms and may be flexible, rigid, semi-rigid, or a combination of these. Indeed, the force-sensitive sensor can measure volume and flow rate arbitrary of the size or shape of the bag/container.

The container can also be a handheld urinal container that a patient can use and then place on the hook 7. The system is suitable for monitoring the urine output of patients who do not have urinary catheters. In this embodiment, the patient urinates into a handheld urinal and then places the urinal on the device for a few seconds. The device converts the weight into a volume and keeps track of the total amount of urine produced. This option can be used to avoid placing a urinary catheter into patients who can use a urinal instead. For example, in the current system whenever a patient uses the handheld urinal a nurse needs to come to the patient's room to record the urine output. Because nurses have busy schedules it is difficult to time things correctly, which can result in urine samples being discarded without being recorded. The device herein makes obtaining urine output data from patients who collect their urine manually with urinals easier and more accurate. This would eliminate the need to place an indwelling catheter in these patients, thereby eliminating the risk of numerous complications including urinary tract infections (UTIs), patient immobility, and urogenital trauma.

The container 10 may be formed with a variety of types of materials known to be suitable for fluid collection bags/containers. For example, the container 10 may be formed with a thin PVC structure, may be a more rigid blow molded plastic container, or may be a container that combines rigid materials and flexible materials. The container 10 can be shaped and sized for various different applications. In some instances, the container 10 will be between about 7 and 15 inches in height and about 1300-3000 mL in volume, (e.g., the container may be about 10 inches in height and about 2000 mL in volume). Preferably, the container 10 has a large enough volume to collect at least the average volume of urine produced by the average patient during urination. Typically, a container volume of at least 2000 mL is desired. Different sizes may be used for different applications, e.g., urine collection from small children may involve smaller sizes than urine collection from adults. Other containers for collecting other fluids such as pericardial or pleural fluids may comprise standard containers for collecting such fluids.

In practice, the container 10 may be designed to fill with fluid from the top or the bottom of the container, e.g., urine 12 can flow from a Foley catheter into tubing 11 associated with the container that empties into the container 10. In one embodiment, fluid flows through tubing connected at the top of the container to fill the container. The fluid generally flows through the tubing/catheter into the container due to gravitational force (although fluid may in some circumstances be drained by other forces, e.g., via a pump or suction). The container 10 may also include a component for removing quantities of fluid, optionally measured, for various testing procedures or merely for emptying the container, such as a drainage tube, drain port, and/or drain valve. Removal of samples from the container may be recorded by the fluid measuring device as a decrease in weight of the fluid, as described herein. The weight decreases recorded by the device may optionally be tabulated and compared to other records regarding fluid removal as a check.

The fluid being measured (e.g., urine 12) flows through the catheter and associated tubing 11 into the collection container 10. Preferably, the inside of the associated tubing/catheter 11 is coated with a hydrophobic coating and/or includes a superhydrophobic pattern design to reduce fluid surface tension on the tubing/catheter. This provides a better emptying mechanism and prevents fluid from being held for too long within the catheter thus affecting the readings.

The fluid being measured (e.g., fluid 12) can also be stationary in a handheld urinal that a patient fills and then places on the hook 7 of the device.

The device needs to be properly positioned so that the measurements are accurate. After initially attaching the device to the supporting apparatus (e.g a bed or stand) using the attachment member, a two-part mechanical algorithm is used to position the device: 1) the device is mounted sufficiently high enough so that the collection container can hang freely from the support member (e.g. hook 7) without touching the floor or any other surface that would prevent the device from measuring the weight of the fluid collection container and fluid therein; and 2) the device is adjusted so that the principal axis of the load cell is directed vertically before any measurements are taken. In the embodiment wherein a bending beam load cell is used, the load cell load bar is oriented level (horizontal) and/or in a configuration that maintains the weight of the fluid collection container aligned with the principal axis of the load cell before any measurements are taken.

With respect to the vertical mechanical support system, it has been found that the accurate fluid output is improved when the device is high enough off the floor so that the fluid collection container does not come into contact with the floor. If this were to occur then the force from the fluid collection container would be transmitted into the floor instead of into the load cell. Therefore, the device must be elevated high enough from the floor. However, the device must not be elevated too high from the floor or it would be above the level of the patient and the urine would not flow by gravity into the fluid collection container.

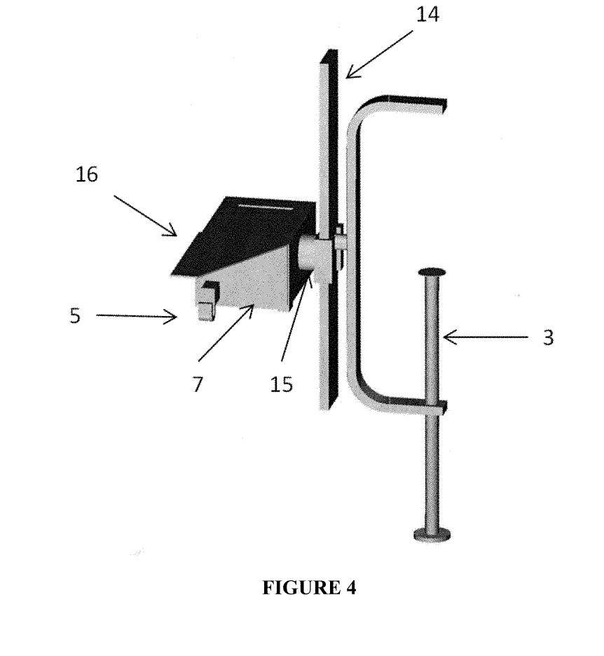

In many instances, the device can be mounted sufficiently high enough to support a free-hanging fluid collection container simply by attaching the device to a supporting apparatus such as a bed rail at the right height. In some instances it may be necessary to use an attachment member with the ability to attach to a supporting apparatus at one height and allow for adjusting the height of the device relative to that height. As discussed below, it may be necessary to adjust the height of the device and/or the height of the load cell in response to a change in the height of the supporting apparatus. FIG. 4 shows an example of an attachment member that can attach to a support apparatus and allow for movement of the device up and down a track. In all instances, the mounting height should be high enough to support a fully filled fluid collection container without touching the floor.

FIG. 4 shows a schematic drawing of the fluid measurement device with an alternative attachment member. In this embodiment, the casing 5 is attached to a bracket 15 that slidably engages a track 14 attached to the C-clamp 3. The C-clamp is attached to a horizontal member of a supporting apparatus such as a bed rail. The bracket 15 can be slid vertically along the track 14 to change the position of the device so that a fluid collection container (not shown) can be suspended from the hook 7 without contacting the floor or other impediment to its ability to hang freely from the hook 7. For example, the device may be moved along the track by a (motorized) pulley system. Once the correct height of the device is achieved, the device may be kept at that height simply by friction between the bracket and the track or by a locking device such as a screw or lever. An alternative height adjustment mechanism may be a vertical rack and pinion system comprising a pair of gears which convert rotational motion into linear motion. A circular gear called a “pinion” engages teeth on a linear “gear” bar called a “rack”. Rotational motion applied to the pinion causes the rack to move relative to the pinion, thereby translating the rotational motion of the pinion into linear motion. In this embodiment, the rack is stationary and the pinion and the attached device travel vertically along the rack. The height of the device can be adjusted manually, or the device can comprise appropriate motors or servos to effect the height adjustment.

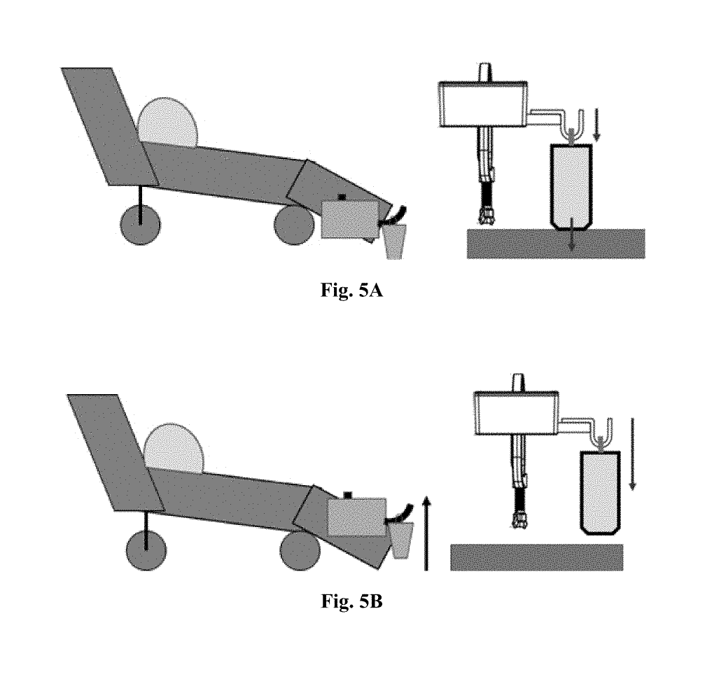

The concept of maintaining a sufficient height for the fluid collection container may be understood with reference to FIGS. 5A and 5B. In FIG. 5A, the hospital bed has been oriented in a sitting configuration, with the fluid measuring device and associated fluid collection bag attached near the foot of the bed. The fluid collection bag is positioned on the hook vertically below the strain bar. The configuration of the bed as a chair has resulted in the device being lowered so that the fluid collection bag is now resting on the floor. As shown by the two small arrows, at least some of the weight (force) of the bag is being supported by the floor and the remaining weight is being supported and measured by the strain gauge. This results in the device registering a lower, inaccurate weight for the fluid bag.

In FIG. 5B, the device has been raised so that the fluid collection bag is no longer on the floor. The entire weight (force) of the bag is supported by the load cell and can be measured accurately by the device. The device could be raised manually by a practitioner, but in preferred embodiments, the microcontroller is configured to use a height adjustment algorithm as shown below to direct the vertical mechanical support system to raise the device, enabling it to obtain accurate measurements of fluid collected in the bag.

In some preferred embodiments, the microcontroller can autonomously determine whether a height adjustment is necessary and make the adjustment. In these embodiments, the vertical mechanical support system is designed to interact with an algorithm built into the device that enables the device to maintain an adequate distance from the floor so that the urine collection container will not come into contact with the floor. The microcontroller can signal motor(s), servo(s) and/or actuator(s) to drive the height adjustment according to the height adjustment algorithm described by example but not limitation below. In particular, the times and height adjustment increments used in the example algorithm can be substituted by other times or height increments. Accordingly, the embodiments described herein achieve more accurate measurements of fluid output.

An example height adjustment algorithm is described as follows. When the device detects a stable fluid weight, e.g, urine weight is stable for period of time, such as 10 minutes, 15 minutes, 20 minute, or 30 minutes, the device's microcontroller sends a message to the motorized pulley system (or other actuator such as a motor to turn the pinion of a rack-and-pinion system) to automatically increase the height of the fluid collection container from the starting point (“Point 0”) by (for example but not limitation) 3 cm (“point +1”). The microcontroller then compares the weight of the collection container at “point 0” and “point +1”. If the weight did not change (such as by less than 5 to 10 g), then the device assumes that “point 0” is an adequate height from the floor and returns to that position. If the weight of the container is higher (greater by at least 5 to 10 g) at “point +1” compared to “point 0” then the device assumes that the fluid collection container was too close to the floor at point 0 and proceeds to increase the height of the load cell to 3 cm above “point +1” (“point +2”). If the weight at point +2 is the same as the weight at point +1, then the device assumes that point +1 was high enough and returns to point +1. If the weight at point +2 is higher than at point +1, the device increases height again until either there is no change in weight between the starting and end points or the maximum point (such as 9 to 15 cm) of the elevation system of the device is reached. Other height adjustment increments are also contemplated (such as 1 cm, 2 cm, or any combination of increments), as are other maximum heights of the elevation system.

The height of the device as set by the vertical mechanical elevation system can be manually adjusted electronically by pressing an “up” or “down” button on the device. Preferably the device can autonomously adjust the height as needed.

Also shown in FIG. 4 is a shelf 16 attached to the casing 5 over the hook 7 to shield the hook 7, and any container suspended from it, from objects that may impinge from above and influence the measurement of fluid.

The device is also adjusted so that the principal axis of the load cell is directed vertically and/or in a configuration that constantly maintains the weight of the fluid collection container aligned with the principal axis of the load cell before any measurements are taken. In the case of a bending beam load cell, in which strain is measured by deflection of the load bar, the load bar should be horizontal (the principal axis of the load cell is directed vertically) for accurate measurements.

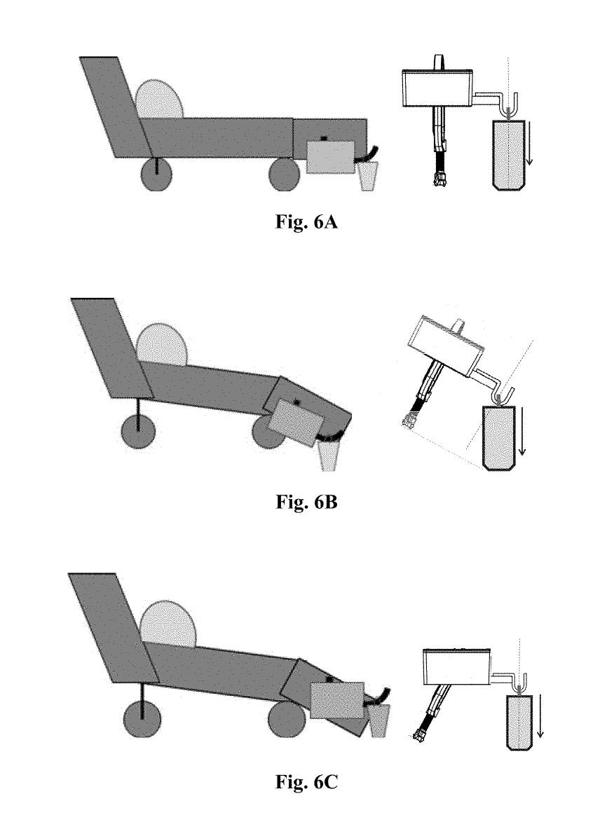

The concept of maintaining the principal axis of the load cell as directed vertically (with the weight of the fluid collection container aligned with the principal axis of the load cell) may be understood with reference to FIGS. 6A to 6C. In FIG. 6A, the hospital bed is in a sleeping configuration, with the fluid measuring device and associated fluid collection container attached near the foot of the bed. The bed is horizontal and the device is oriented so that it is also level. The load bar of the load cell is also level, and the principal axis of the load cell is directed vertically as indicated by the dashed line. The fluid collection bag is positioned on the hook below the strain bar. The weight (force) of the bag, as indicated by the arrow, is directed vertically and aligned with the principal axis of the load cell, so an accurate measurement of weight can be taken by the device.

The adjustable, rotational mechanical support is operatively connected to a level-determining device such as a gyroscope, which is configured to monitor the position of the device in space to ensure that the principal axis of the load cell remains directed vertically. With respect to the level-determining device and rotational mechanical support, when the device is attached to a hospital bed and the load cell is fixed to the device with a fluid collection container suspended from the device, the fluid collection container must remain aligned with a stable axis relative to the device. Under circumstances where the angle of the hospital bed is adjusted this will cause the angle between the principal axis of the load cell and fluid collection container to change (the weight of the container will change from an axial load to an inclined load). The gyroscope can detect these changes and then signal to the rotational mechanical support system to rotate the device so that it remains parallel to the floor and maintaining the principal axis of the load cell directed vertically.

In FIG. 6B, the hospital bed is in a chair or sitting configuration and the foot of the bed is diagonal relative to the floor, not horizontal. The device attached to the bed is no longer level or horizontal and the principal axis of the load cell is not directed vertically (indicated by the dashed line). The weight (force) of the bag (indicated by the arrow) is not aligned with the principal axis of the load cell, resulting in an inclined load, so the weight of the bag would not be measured accurately.

FIG. 6C shows the bed in the chair configuration with the device attached. In FIG. 6C, the level determining mechanism and the rotational mechanical system have reoriented the device so that it and the attached strain bar are level (horizontal). The principal axis of the load cell is restored to be directed vertically (indicated by the dashed line) and the weight of the fluid bag (indicated by the arrow) is aligned with the principal axis of the load cell, allowing an accurate measurement of the weight of the fluid bag to be obtained.

A bull's eye spirit level can be provided on the exterior of the device to facilitate leveling of the device, or more particularly the load bar of the bending beam load cell of the device. Built-in electronic tilt sensors and/or inclinometers can be used to determine the degree of level. Common tilt sensors and inclinometers include a gyroscope, accelerometer, liquid capacitive, electrolytic, gas bubble in liquid, and pendulum sensors. Once the initial attachment of the device to the supporting apparatus is accomplished, adjustments of level can be accomplished by the rotational adjustment mechanism rotating the device (or at least the load cell) on an axis orthogonal to the principal axis of the load cell so that its principal axis is directed vertically.

This can be accomplished using small devices such as screws, shims, axles or gears to change the orientation of the load bar relative to the attachment member. The electronic sensors can be integrated with the microcontroller to provide information to the practitioner to enable leveling the device, such as by displaying leveling instructions on the display screen. Preferably, the electronic sensors are integrated to the microcontroller to enable it to determine the need for an adjustment and carry out the adjustment autonomously (i.e. without practitioner intervention).

Alternatively, a combination of Micro-Electro-Mechanical Systems (MEMS) microelectronics, microsensors and microactuators may be used by the electronics package to level the load bar. Microsensors and microactuators are appropriately categorized as transducers. In the case of microsensors, the device typically converts a measured mechanical signal such as tilt into an electrical signal, which can be converted back into a mechanical force by a microactuator to level the load bar of the device.

In some preferred embodiments, the microcontroller can autonomously determine whether a rotational adjustment is necessary and make the adjustments. In these embodiments, the rotational mechanical support system is designed to interact with an algorithm built into the device that enables the device to maintain an orientation to keep the principal axis of the load cell directed vertically and the weight of the fluid collection container aligned with the principal axis of the load cell. The microcontroller can signal motor(s), servo(s) and/or actuator(s) to drive the rotational adjustment according to the rotational adjustment algorithm. Accordingly, the embodiments described herein achieve more accurate measurements of fluid output.

In some instances, the electronics may be configured to determine a need to signal a practitioner via a notification code and/or alarm that the device is not sufficiently level to take accurate measurements and cannot re-level itself.

Whenever the mechanical elevation or mechanical rotation systems are moving, the device uses a built-in force sensor to detect whether it is encountering resistance. If it encounters a resistance above a specific force threshold then it assumes there is an object in the path of the device and stops moving. The device can generate a local user alarm in these situations.

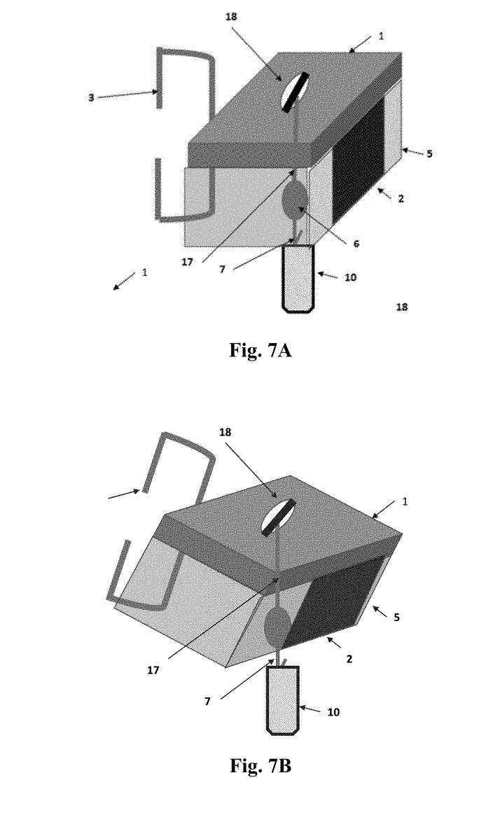

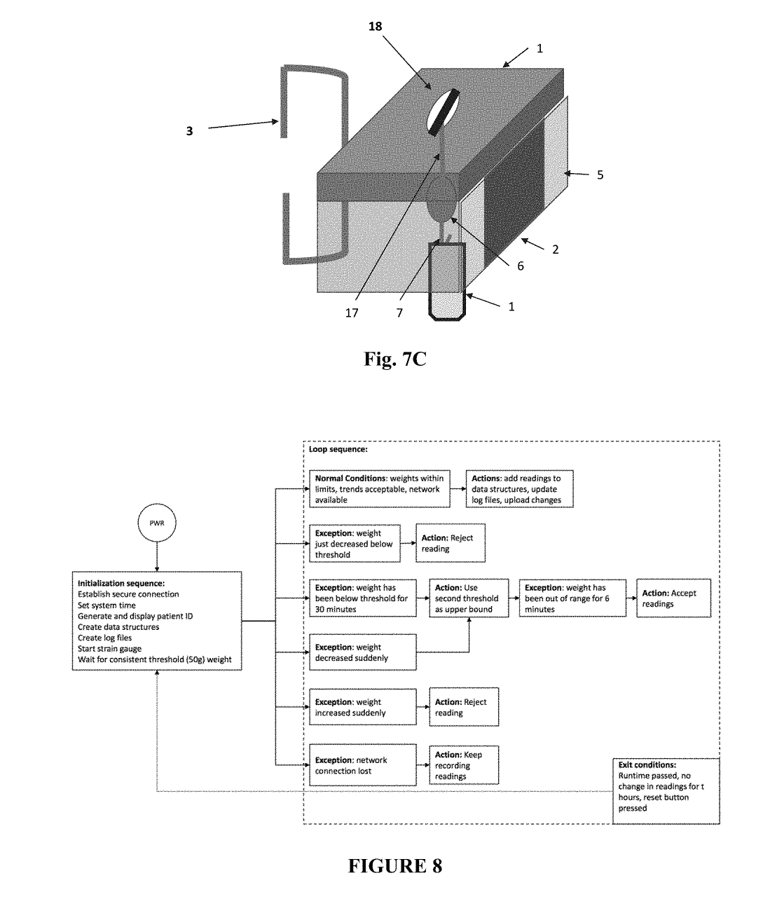

In another embodiment, shown schematically in FIGS. 7A to 7C, the device comprises a tension load cell 6 such as a Tension Compression Mini Load Cell suspended from a motorized pulley system 18 by a wire or tape 17. The fluid collection container 10 attaches underneath the free hanging load cell by a hook 7. In this embodiment, the load cell measures tension in the vertical axis due to the weight of the fluid collection container. The vertical direction of the principal axis of the load cell is automatically maintained because the weight of the load cell and attached fluid collection container function as a plumb-bob or plummet. Because the load cell is suspended freely from the device the weight (force) of the fluid collection container remains automatically oriented along the principal axis of the load cell at all times, even in circumstances where the supporting apparatus (e.g. hospital bed rail) and the attached device are positioned at an angle to the floor (FIG. 7B).

The device is attached to the supporting apparatus (e.g. hospital bed rail) via a clamp 3 or straps and the suspended load cell monitors the weight of the fluid collection container 10 hanging underneath. The motorized pulley system 18 is used to automatically adjust the height of the load cell so that the fluid collection container remains freely suspended off the floor by retracting or extending the length of the wire or tape 17 suspended from the pulley according to the height adjustment algorithm described above. As shown in FIG. 7C, the load cell and fluid collection container have been raised relative to the casing 5 by shortening the wire or tape 17.

The casing 5 in this embodiment is configured to have an opening in the bottom and enclosed sides to provide a cavity in the lower portion of the casing in which the load cell is suspended. The casing sides protect the load cell from external forces (such as blankets, pillows, clinical staff or visitors inadvertently moving the load cell) while still allowing for the load cell to hang vertically if the casing of the device is tilted. The device also includes a cover (not shown) that protects the motorized pulley system from external objects. The device also contains space for a screen 2 for displaying and entering data.

In the embodiment above, the load cell 6 is moved up and down by the motorized pulley system 18 and its position changes relative to the rest of the device, while the casing of the device stays in one position relative to the supporting apparatus. In yet another embodiment, the device comprises a tension load cell as described above, but it is freely hanging from a fixed pivot point in the device. In this embodiment, the position of the entire device (not just the load cell) is adjusted vertically along a track similar to that shown in FIG. 4, such that it is raised relative to where the attachment member is attached to the supporting apparatus. Raising the device along the track can be effected by a motorized pulley system controlled by the microcontroller according to the previously described height adjustment algorithm. Alternatively, the device can be raised or lowered using a rack and pinion system.

The fluid meter can easily measure the duration of urination because the calculation of increased urine volume will suddenly change when the first urine enters the fluid container, and will also change by a significant amount when the last of the urine enters the collection container and the measurements no longer increase. The frequency of measurements taken over time (such as for example from 10 to 60 seconds between measurements) provides a good indication of the start and stop times for urination.

The fluid measurement device is preferably able to measure a sufficient range to simulate urine output, be precise in measurements, and provide repeatable results to within an error of no more than +/−5 mL. More preferably, the sensor will be able to provide repeatable results to within an error of no more than +/−2 mL.

Structural variations in the fluid meter, including structural variations in the force sensor (e.g. load cell), are possible without straying from the general principles described herein, e.g., force-based sensing principles such as strain forces from the weight of the collection container on the sensor hook.

Structural variations in the rotational and vertical mechanical systems are possible without straying from the general principles described herein, e.g., mechanical systems to maintain the principal axis of the load cell directed vertically, and the fluid collection container suspended sufficiently off the floor.

The measured forces from the sensor may be correlated to volume using software/firmware in the microcontroller, such as by machine learning. Examples of machine learning include, but are not limited to, linearization and/or curve-fitting procedures, and decision trees. A decision tree enables the device to capture real changes in fluid volume and ignore noise. The algorithm allows for a high degree of accuracy and precision. The software/firmware may then be programmed with the relationship in order to calculate, for any given force measurement, a value for liquid volume in the container. The software/firmware may also be programmed to track the volume level over time to calculate flow rate. The software/firmware may also signal that the volume level, flow rate, and any other measured/calculated parameters be displayed on or transferred to a monitor, computer, smart phone, and/or other device. The parameters may be continuously calculated, updated, and displayed in real time, e.g., during fluid collection. The software/firmware may also be programmed to accomplish other purposes/functions, including those discussed elsewhere herein.

Notably, the controller may be programmed to measure fluid volume and flow according to an algorithm that enables the continuous change in mass of a fluid bag over time to be converted to a fluid volume automatically without the need to manually tare the system. The algorithm is designed to recognize the presence of a stable fluid bag and account for missing measurements that occur if the fluid bag is moved from the device or if fluid is removed from the bag. The algorithm can also be used to quantify the amount of fluid volume in a handheld urinal by having the patient place the urinal on the scale and the device records the amount of fluid in the container.

The pre-programmed algorithm is necessary because the busy, harsh environment in hospitals and long-term care facilities makes it considerably challenging to assess fluid volumes based on the changing weight of a fluid collection container. For example, if the position of the bag changes due to the patient moving, nurse handling, or being jostled by patients, visitors or staff, then the observed weight may change as the bag resettles. The device needs to have the capability to identify and ignore this noise without missing important information such as a patient rapidly producing urine in response to medication. Additionally, when a patient is transported to another location such as for a test or therapy, the fluid collection container is transported with the patient while the device remains attached to its original location, such as the original hospital bed. The device needs to determine how to incorporate the weight of the bag when the patient returns from the test or therapy. For example, the weight may be higher since more fluid will have been produced, or the weight can be lower, if the bag was emptied or a sample removed. The device will also need to decide how to register the absent data.

The load bar/amplifier continuously measures the weight of the fluid collection container. The raw data is analyzed by the microcontroller, which applies an algorithm to assess the validity of each data point. The algorithm is able to calculate data that remains highly accurate (such as greater than 95%) in real clinical practice. The algorithm is based on a decision tree, machine learning approach summarized in FIG. 8.

The algorithm uses a running average, standard deviation, and comparison to previously accepted values to determine fluid output. The algorithm relies on the following parameters:
1) Repeat measurements are made at regular intervals;
2) A series of measurements are averaged together;
3) This average must meet a series of criteria including, but not limited to, minimum value cut off and a minimum standard deviation cutoff in order to be accepted;
4) The new accepted average is then compared to the previous accepted average;
5) Based on whether the new accepted average is higher or lower than the previous accepted average determines whether the device adds new data to the cumulative or restarts from a new baseline.
6) These cutoff values can be adjusted by the algorithm based on analysis of current fluid output flow rates.

Summarized below is an example of how the algorithm is applied to the measurement of fluid volume/flow from a patient. In this example the average weight cutoff is based on the weight of a standard commercial urine collection container. The standard deviation cutoff is based on assumptions of maximal rates of continuous urine output for patients with urine catheters on diuretic medications. Situations where a patient leaves for a test are addressed by assuming that if the weight of the bag is higher than when the patient left then more fluid is present and this data should be added as new weight. However, if there is less weight than the previous accepted volume then no new volume should be added and the device should consider this to be a new baseline. The algorithm below is for example but not limitation, so other values may be used in the algorithm. For example, the number of measurements used in the running average may vary from 3 to 10. Other time increments are also contemplated (such as measurement intervals from 10 seconds to 60 seconds and/or waiting periods from 1 to 10 minutes to allow for settling). Weight cutoffs may also be varied, such as thresholds from 20 g to 100 g for the average to be accepted. Standard deviation thresholds may vary from 3 to 15. In some embodiments, the algorithm may be programmed into the device with default settings, such as the ones included in the example algorithm below. In some embodiments, the settings in the algorithm may be modified as desired by a practitioner to allow for fluid measurements to be customized for a given patient, monitoring protocol, etc. Modifying the settings of the algorithm can be accomplished by inputting the desired settings locally using the touch screen of the device and/or from a remote device such as a work station or central records facility using the WiFi transceiver.

### I. Zero

1) Device is turned on;
2) Waits 3 minutes before making any measurement (to allow for things to settle down);
3) Makes 6 measurements each 20 seconds apart; and
4) The measurements are averaged and a standard deviation is calculated.
5) If the average is less than 50 g, then all 6 measurements are rejected (assumption that bag is not on scale) and returns to step 3;
6) If average is greater than 50 g then:
   a. If the standard deviation is less than 9 then the device records that weight as zero;
   b. If the standard deviation is greater than 9 the device rejects those 6 measurements and returns to step 3.

### II. Measurements

1) Makes 6 measurements each 20 seconds apart;
2) The measurements are averaged and a standard deviation is calculated;
3) If the average is less than 50 g then reject all 6 measurements are rejected (assumption that bag is not on scale) and returns to step 2;
4) If the average is greater than 50 g then:
   a. If the standard deviation is less than 9 than the device accepts the average weight;
   b. If the standard deviation is greater than 9 the device rejects those 6 measurements and returns to step 3;
5) The average weight is subtracted from the previous average weight to calculate a new weight;
   a. If the new interval weight is positive then the new weight is added to the previous cumulative weight;
   b. If the new weight is negative then the device records this weight as the new zero and the cumulative volume remains unchanged.

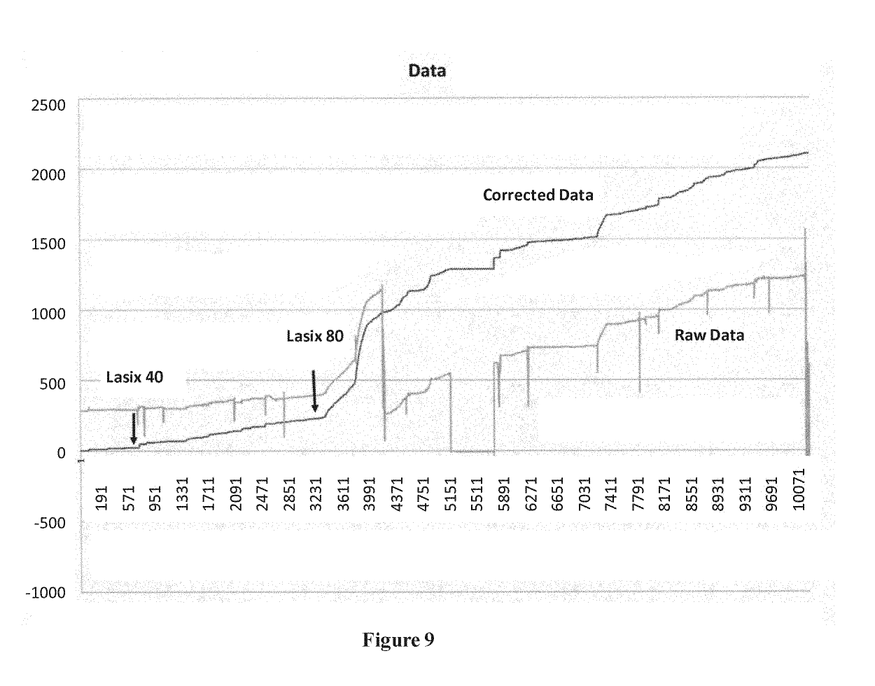

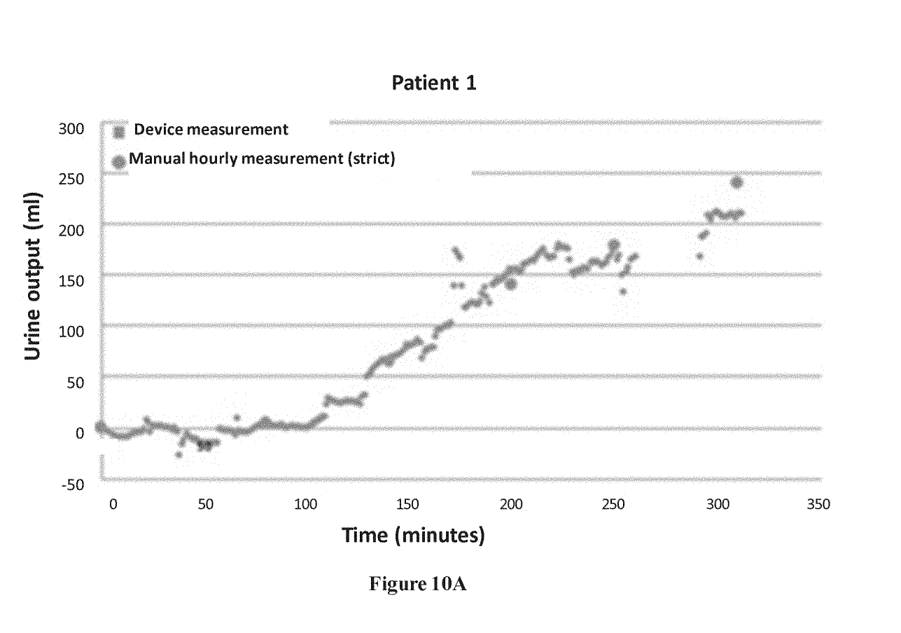

Situations where the fluid collection container is moved, jostled or removed during a measurement run can be identified by the device in the pattern of weight readings. For example, a rapid fluctuation or “spike” in the weight measurements may indicate that the container was jostled. A series of such fluctuations may indicate that the container was being moved, such as during patient movement or transport. A rapid drop in weight followed by a period of no weight change and then a rapid increase in weight may indicate that the container was removed from the device. FIG. 9 shows raw data collected by the device compared to corrected data where the non-accepted readings are ignored by the algorithm. “Spikes” in the raw data indicate movements of the collection container and the weight readings for those points are not included in the total volume output in the corrected data. The drop to zero indicates when the container was removed from the device. Also indicated on the chart are times when Lasix diuretic was administered, which led to increased fluid output.

Based on the data collected the device can also determine the optimal method for displaying the data on the local display screen or the rate of which to send data to the online cloud or EMR.

Optionally, the device may include a “pause/resume” feature that allows a practitioner to temporarily interrupt the algorithm during a period when disruption of accurate measurement is likely, such as during patient movement or transport, or draining of the fluid collection container. When the practitioner activates the pause feature, the device stops taking measurements, but does not reset the cumulative output measure to zero or delink the device from the data server. When the practitioner resumes the algorithm, the algorithm will run through steps in the “Zero” portion of the algorithm to ensure that a collection container is present and that the device is stable before resuming measurements. The new measurements will be added to the previous cumulative total. If the device detects a series of erratic readings, the algorithm may automatically enter the pause mode followed by automatically entering the resume mode, allowing it to recheck stability before taking new measurements. The device may also automatically resume measurements if the pause feature is activated for more than a predetermined period, so that fluid measurement is not discontinued for too long. Even without a pause/resume feature, the algorithm used by the device is capable of recognizing and correcting for excursions such as described above to provide an accurate cumulative total of the weight measurements.

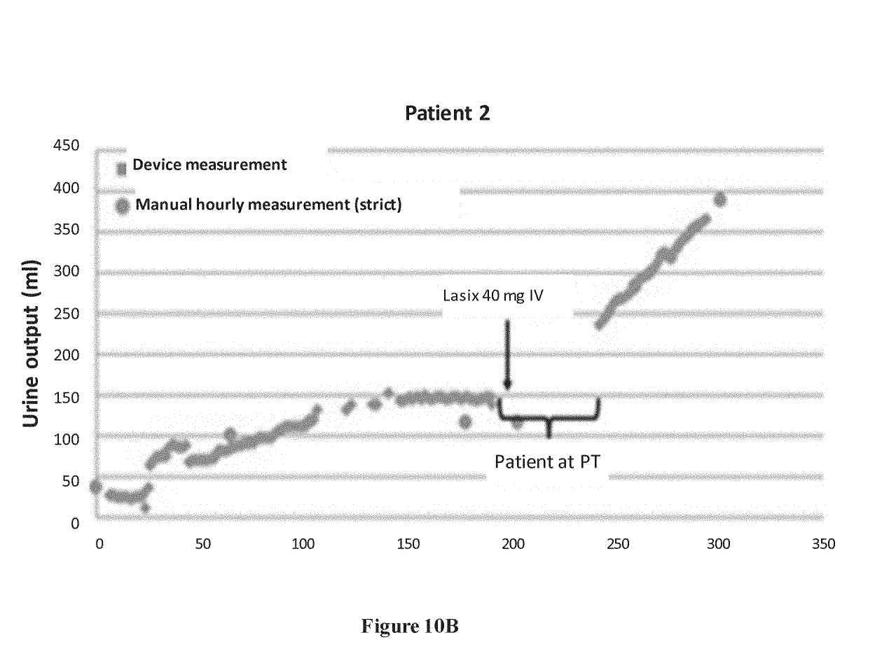

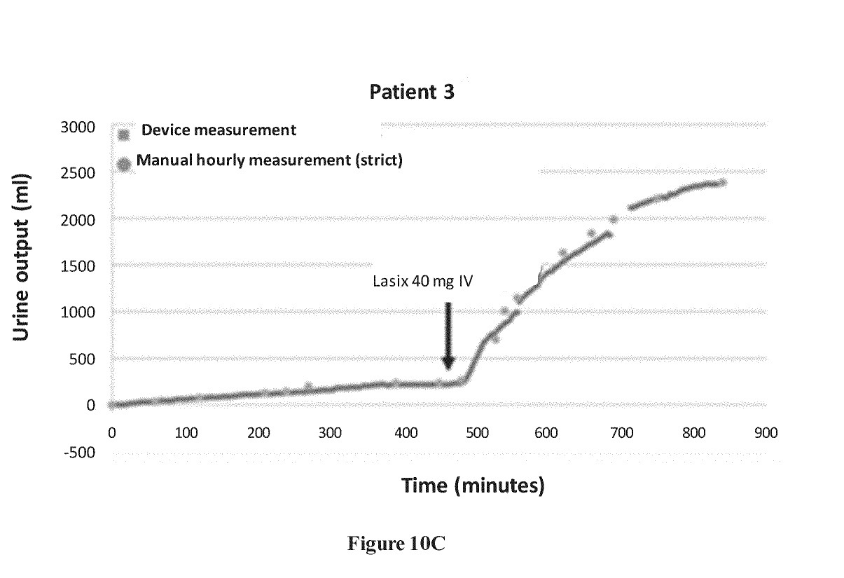

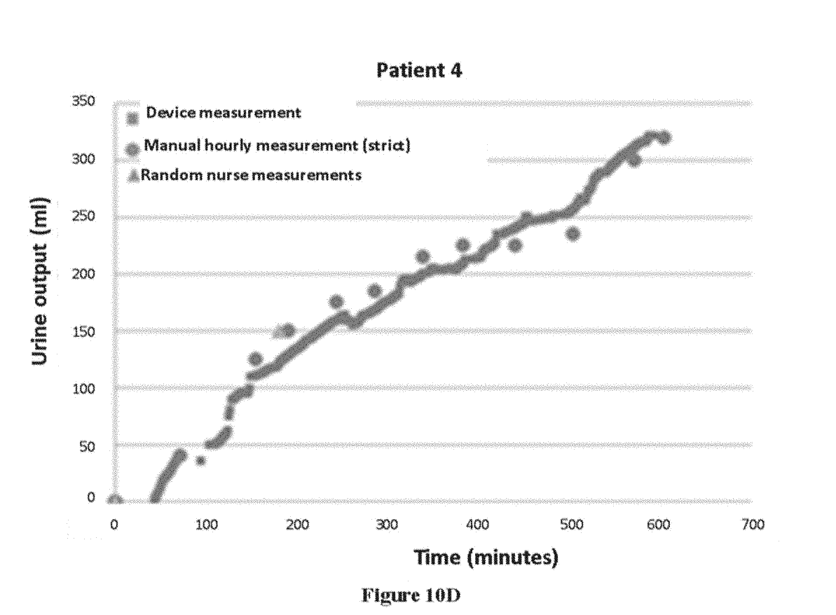

Preliminary clinical data from patient tests demonstrated the capabilities of the device (FIGS. 10A to 1D). Patient 1 (FIG. 10A) demonstrates a five-hour experiment whereby the accuracy of the system was practically identical to that of strict, manual, hourly, measurements by a trained clinician. Patients 2 and 3 (FIGS. 10B and 10C) demonstrate the device's ability to detect an increase in urinary output (UOP) following treatment with an intravenous diuretic agent (furosemide). Since the device works based on the weight of the urine bag no data was lost when patient 2 went for physical therapy (PT) and the algorithm was able to appropriately represent the data once the patient was placed back in bed and the collection bag back on the device (FIG. 10B). The breaks in the graph for patient 3 (FIG. 10C) occurred because the urine collection bag was mistakenly left off the device by the nurse after the patient had been repositioned. The algorithm was able to analyze the data and prevent information from being lost once the bag was placed back on the scale. Patient 4 (FIG. 10D) compares ten hours of monitoring using the device against strict, manual, hourly, measurements by a trained clinician, and against measurements recorded by the ICU nurse during the course of usual care. In this test the device was superior to a trained ICU nurse (nurse's measurements depicted as triangles). While these studies were performed under artificially controlled clinical settings these results suggest that the device described herein can accurately monitor UOP, and is as accurate as strict, manual, hourly measurements by a trained clinician, and possibly even superior to current standard of care.

Any data measured by the fluid meter (e.g., volume and flow rate data) can be processed (using a pre-programmed machine learning based algorithm) and displayed on the device or may be sent to another device or computer (e.g., a desktop computer, a laptop computer, a smart phone, etc.) to collect, process, and/or store the data for review and tracking. The other device or computer may include a web-based server, secondary digital display and/or processor such as a bedside monitor, a ward nursing station, a clinician's personal computer or work station, a centralized electronic medical records server, or the like. A finger-type card edge connector may optionally be included as part of the fluid meter. The finger-type card edge connector provides a means of connecting fluid meter to another device or computer, and provides a means for communicating data from the microcontroller to the connected device or computer.

Various devices, systems, or means for connection/communication between the fluid meter and other devices or computers are also possible. For example, the fluid meter may include a USB port, an Ethernet port or any other data transmission port and/or may be tethered to a device or computer through a wired connection. Alternatively, the fluid meter may include a wireless transmitter or transceiver (e.g., Zigbee, etc.) to transmit data wirelessly. In one embodiment, short range radiofrequency (RF) principles may be used. Some short range RF protocols that can be used are referred to as “Bluetooth.” Wireless 802.11 communication principles and/or similar communication principles may also be used. The fluid meter or the device or computer with which it communicates may optionally be connected to a network (e.g., the internet or a local network) and the data may be shared with and/or processed by other devices or computers connected to the network. As described above, the volume and flow data may be displayed and/or sent in real time as it is generated. The data may be preferably linked with a unique patient identifier that provides security and privacy to the information.

In one embodiment, multiple fluid meters each connected to a different patient are configured to transmit data to the same computer or network. This allows tracking and/or comparing data from multiple patients at a single location. Software associated with each fluid meter can be programmed to transmit the measured data with a unique identifier to distinguish the data transmitted by one fluid meter from the data transmitted by each of the other fluid meters.

The above fluid monitoring systems have generally been described as being applied to a urine meter(s) or urine monitoring systems; however, the principles described may be applied to other types of fluid measurement or monitoring systems, i.e., not involving urine. Further, the features described in one embodiment may generally be combined with features described in other embodiments.

Components of the apparatuses, devices, systems, and methods described herein may be implemented in hardware, software, or a combination of both. Where components of the apparatuses, devices, systems and/or methods are implemented in software, the software (e.g., software including algorithms/calculations discussed above) may be stored in an executable format on one or more non-transitory machine-readable mediums. Further, the algorithms, calculations, and/or steps of the methods described above may be implemented in software as a set of data and instructions. A machine-readable medium includes any mechanism that provides (e.g., stores and/or transports) information in a form readable by a machine (e.g., a computer). For example, a machine-readable medium includes read-only memory (ROM); random-access memory (RAM); magnetic disk storage media; optical storage media; flash memory devices; DVD's, electrical, optical, acoustical or other form of propagated signals (e.g., carrier waves, infrared signals, digital signals, EPROMs, EEPROMs, FLASH, magnetic or optical cards, or any type of media suitable for storing electronic instructions. The information representing the apparatuses and/or methods stored on the machine-readable medium may be used in the process of creating the apparatuses, devices, systems, and/or methods described herein. Hardware used to implement the disclosed subject matter may include integrated circuits, microcontrollers, FPGAs, digital signal controllers, and/or other components.

While the disclosed subject matter has been described in terms of particular variations and illustrative figures, those of ordinary skill in the art will recognize that the disclosed subject matter is not limited to the variations or figures described. In addition, where methods and steps described above indicate certain events occurring in certain order, those of ordinary skill in the art will recognize that the ordering of certain steps may be modified and that such modifications are in accordance with the variations of the disclosed subject matter. Additionally, certain of the steps may be performed concurrently in a parallel process when possible, as well as performed sequentially as described above. Therefore, to the extent there are variations of the disclosed subject matter, which are within the spirit of the disclosure or equivalent to the disclosed subject matter found in the claims, it is the intent that this patent will cover those variations as well.

1. A fluid monitoring device, comprising
(a) a support and measurement assembly comprising a support member configured to support a container for collecting fluid and a sensor in operational communication with the support member wherein the sensor is configured to measure the weight of the container and fluid collected therein and provide an electrical signal proportional to the measured weight; and
(b) a microcontroller in electrical connection with the sensor, configured to signal the sensor to weigh the container periodically, receive the electrical signal from the sensor and to use data from the sensor to calculate a volume of the fluid collected in the container using a pre-programmed machine learning based algorithm.

2. The fluid monitoring device according to claim 1, wherein the support and measurement assembly comprises a strain gauge load cell to measure the weight and provide the electrical signal.

3. The fluid monitoring device according to claim 1, wherein the load cell comprises a bending beam load cell or a tension load cell capable of translating up to 10 kg of force with an accuracy of 1-2 g into an electrical signal.

4. The fluid monitoring device according to claim 1, wherein the microcontroller includes software programmed to analyze the raw weight of the fluid collection container and then convert this data into a volume using the machine learning based algorithm.

5. The fluid monitoring device according to claim 1, wherein the algorithm uses at least one of a linearization procedure or a curve fitting procedure.

6. The fluid monitoring device according to claim 1, wherein the algorithm includes a decision tree format.

7. The fluid monitoring device according to claim 1, wherein the algorithm converts the changing weight of the container into a volume while filtering out signal noise from the clinical environment.

8. The fluid monitoring device according to claim 1, wherein the algorithm assesses the validity of each data point.

9. The fluid monitoring device according to claim 1, wherein the algorithm comprises the parameters of:
1) repeating measurements at regular intervals;
2) averaging a series of measurements to provide a running average;
3) determining that the running average meets a minimum value cut off and a minimum standard deviation cutoff in order to be accepted;
4) comparing the new accepted average to a previously accepted average;
5) determining whether the device adds new data to the cumulative or restarts from a new baseline based on whether the new accepted average is higher or lower than the previous accepted average; and
6) adjusting the cutoff values based on analysis of current fluid output flow rates.

10. The fluid monitoring device according to claim 1, wherein the algorithm uses a running average, standard deviation and comparison to previously accepted values to determine fluid output.

11. The fluid monitoring device according to claim 10, wherein (1) the running average and a standard deviation are calculated based on 3 to 10 measurements with measurement intervals from 10 seconds to 60 seconds, and wherein (a) if the running average is less than a threshold from 20 g to 100 g, then all measurements are rejected; (b) if the running average is greater than the threshold from 20 g to 100 g and the standard deviation is greater than a threshold from 3 to 15, then all measurements are rejected; or (c) if the running average is greater than the threshold from 20 g to 100 g and the standard deviation is less than the threshold from 3 to 15, the average weight is accepted; (2) the accepted average weight is subtracted from a previous accepted average weight to provide a new interval weight; wherein (a) if the new interval weight is positive, then the new interval weight is added to the cumulative weight; or (b) if the new interval weight is negative, then the device records the new interval weight as a new zero and the cumulative weight is unchanged.

12. The fluid monitoring device according to claim 1, further comprising a wireless transceiver for transmitting the volume measurement to a separate device.

13. The fluid monitoring device according to claim 1, wherein the microcontroller includes software programmed to transmit the volume measurement with a unique identifier to distinguish the volume transmitted by the fluid monitoring system from data transmitted by other monitoring systems.

14. The fluid monitoring device according to claim 1, wherein the microcontroller includes software programmed to transmit the volume measurement with a programming language compatible with an electronic medical record system.

15. The fluid monitoring device according to claim 1, further comprising a display screen that can display information.

16. The fluid monitoring device according to claim 15, wherein the information is selected from the group consisting of measured fluid volume, flow rate, function code, identification code, instructions, and any combination thereof.

17. The fluid monitoring device according to claim 1, further comprising a touch screen configured for inputting information into the device.

18. The fluid monitoring device according to claim 1 further comprising an attachment member configured to attach the device to a supporting apparatus.

19. The fluid monitoring device according to claim 18, wherein the attachment member comprises at least one clamp, hook, bracket or strap.

20. The fluid monitoring device according to claim 18, wherein the device includes a mechanism for implementing a mechanical algorithm for vertically positioning the device so that 1) the device is mounted sufficiently high enough so that the collection container can hang freely from the support member without touching any surface that would prevent the device from measuring the weight of the fluid collection container and fluid therein; and 2) the device is adjusted rotationally so that the principal axis of the load cell is directed vertically.

21. The fluid monitoring device according to claim 20, wherein the mechanism to position the device vertically comprises a bracket slidably engaged to a track configured to allow the device to move vertically along the track, or a vertical rack and pinion system.

22. The fluid monitoring device according to claim 20, wherein the device comprises at least one level-determining device selected from the group consisting of a gyroscope, bull's eye spirit level, inclinometer, electronic tilt sensor, accelerometer, liquid capacitive level, electrolytic level, gas bubble in liquid level, pendulum level, and micro-electro-mechanical system level.

23. The fluid monitoring device according to claim 20, wherein the mechanism to adjust the device rotationally comprises a screw, shim, gear or axle.

24. The fluid monitoring device according to claim 1; wherein the device is removably attached to (c) a container for collecting a fluid.

25. The fluid monitoring device of claim 24, wherein the container is a urine collection container, pericardial fluid collection container, pleural fluid collection container, or a handheld urinal.

26. The fluid monitoring device of claim 24, wherein the container is operationally connected to a fluid collecting device selected from the group consisting of a urine catheter, pericardial drain, chest tube, and Jackson-Pratt drain.

27. The fluid monitoring device of claim 24, wherein the fluid measured is urine, pericardial fluid, pleural fluid, blood, or blood serum.

28. The fluid monitoring device of claim 24, wherein data obtained by the device is displayed on the display screen of the device, or transmitted via Wi-Fi over the interne to a dedicated local server or a cloud based system and accessed from a secure website, or directly integrated into an electronic medical record system.

29. A method of measuring a fluid volume, comprising: providing a fluid measuring device according to claim 1; contacting a fluid collection container with the fluid measuring device in a manner configured to enable weighing of the container and fluid collected therein; collecting a fluid in the fluid collection container; measuring the weight of the fluid collection container and the fluid collected therein and converting the weight measurement to an electrical signal proportional to the weight using the sensor; sending the electrical signal to the microcontroller; and calculating a volume of the fluid as it collects in the container based on data from the sensor.

30. The method of claim 29, further comprising implementing a mechanical algorithm for positioning the device vertically so that 1) the device is mounted sufficiently high enough so that the collection container can hang freely from the support member without touching any surface that would prevent the device from measuring the weight of the fluid collection container and fluid therein; and 2) the device is adjusted rotationally to be in a configuration wherein the principal axis of the load cell is directed vertically and maintains the weight of the fluid collection container aligned with the principal axis.

31. The method of claim 29, wherein calculating the volume of the fluid comprises using the machine learning based algorithm.

32. The method of claim 31, wherein the algorithm uses at least one of a linearization procedure or a curve fitting procedure.

33. The method of claim 31, wherein the algorithm includes a decision tree format.

34. The method of claim 31, wherein the algorithm converts the changing weight of the container into a volume while filtering out signal noise from the clinical environment.

35. The method of claim 31, wherein the algorithm assesses the validity of each data point.

36. The method of claim 31, wherein the algorithm comprises the parameters of:
1) repeating measurements at regular intervals;
2) averaging a series of measurements to provide a running average;
3) determining that the running average meets a minimum value cut off and a minimum standard deviation cutoff in order to be accepted;
4) comparing the new accepted average to a previously accepted average;
5) determining whether the device adds new data to the cumulative or restarts from a new baseline based on whether the new accepted average is higher or lower than the previous accepted average; and
6) adjusting the cutoff values based on analysis of current fluid output flow rates.

37. The method of claim 31, wherein the algorithm uses a running average, standard deviation and comparison to previously accepted values to determine fluid output.

38. The method of claim 37, wherein (1) the running average and a standard deviation are calculated based on 3 to 10 measurements with measurement intervals from 10 seconds to 60 seconds, and wherein (a) if the running average is less than a threshold from 20 g to 100 g, then all measurements are rejected; (b) if the running average is greater than the threshold from 20 g to 100 g and the standard deviation is greater than a threshold from 3 to 15, then all measurements are rejected; or (c) if the running average is greater than the threshold from 20 g to 100 g and the standard deviation is less than the threshold from 3 to 15, the average weight is accepted; (2) the accepted average weight is subtracted from a previous accepted average weight to provide a new interval weight; wherein (a) if the new interval weight is positive, then the new interval weight is added to the cumulative weight; or (b) if the new interval weight is negative, then the device records the new interval weight as a new zero and the cumulative weight is unchanged.

39. The method of claim 29, wherein the container is a urine collection container, pericardial fluid collection container, pleural fluid collection container, or handheld urinal.

40. The method of claim 29, wherein the container is operationally connected to a fluid collecting device selected from the group consisting of a urine catheter, pericardial drain, chest tube, and Jackson-Pratt drain.

41. The method of claim 29, wherein the fluid measured is urine, pericardial fluid, pleural fluid, blood, or blood serum.

42. The method of claim 29, further comprising calculating a flow rate of the fluid as it collects in the container based on data from the sensor.

43. The method of claim 29, wherein data obtained by the device is displayed on the display screen of the device, or transmitted via Wi-Fi over the internet to a dedicated local server or a cloud based system and accessed from a secure website, or directly integrated into an electronic medical record system.

44. A portable reusable device for measuring fluid output from a patient, comprising:
a housing including a top and bottom surface and sidewalls defining an enclosed space, the housing including a subsystem including a processor configured to measure fluid output expelled from a patient;
a fastener to removably attach the device to an object; and
an adjustable, rotational mechanical support physically engaged to the housing and operatively engaged to a gyroscope, wherein the gyroscope monitors the device position and further wherein the mechanical support rotates the device position upon feedback related to the gyroscope monitoring.

45. The device of claim 44, wherein the processor is capable of recording change in weight of a fluid expelled from a patient over time.

46. The device of claim 44, wherein the surface or sidewall of the housing further includes a display.

47. The device of claim 46, wherein the display is a digital display that has an output for information relating fluid expelled from the patient.

48. The device of claim 47, wherein the information is urine output.

49. The device of claim 47, wherein the information includes data to determine diuretic dosing information, patient weight information, BUN/Cr information, or potassium ion information.

50. The device of claim 44, wherein the subsystem further includes memory for storing measurements made by the processor.

51. The device of claim 44, the subsystem further includes a transceiver for sending information to and receiving information from a remote location.

52. The device of claim 51, wherein the transceiver is capable of sending fluid measurement information assessed by the processor to a remote location.

53. The device of claim 44, wherein the fluid is selected from the group consisting of urine, pericardial fluid, pleural fluid, blood, or blood serum.
